(19) United States
(12) Patent Application Publication
(10) Pub. No.: US 2021/0349529 A1
(43) Pub. Date: Nov. 11, 2021

(54) AVATAR TRACKING AND RENDERING IN VIRTUAL REALITY

(71) Applicant: MVI Health Inc., Alameda, CA (US)

(72) Inventors: Hans Peter Winold, Berkeley, CA (US); Andrew Taylor Langley, Alameda, CA (US)

(21) Appl. No.: 17/314,506

(22) Filed: May 7, 2021

Related U.S. Application Data
(60) Provisional application No. 63/022,186, filed on May 8, 2020.

(51) Int. Cl. G06F 3/01; G06T 13/40

(52) U.S. Cl. CPC G06F 3/012; G06T 13/40; G06F 3/014

(57) ABSTRACT

Virtual Reality systems may be used in healthcare and therapy, e.g., focusing on physical and neurorehabilitation. For instance, victims of brain injury may seek treatment to improve, e.g., range of motion, balance, coordination, joint mobility, flexibility, posture, endurance, and strength. VR systems can be used to entertain and instruct patients in their movements while recreating practical exercises to further therapeutic goals. Patient movement data during physical therapy sessions may be valuable to patients and healthcare practitioners. The system may comprise a plurality of body sensors, a VR headset, and a supervisor tablet. The disclosed system may facilitate translation between real world coordinates and virtual world coordinates, assigning sensors to body parts, measuring range of motion of joints and limbs, correcting sensor orientation, recording and presenting therapy data, and generating and animating a 3-D virtual reality avatar in a virtual world to perform activities, among other benefits.

### CLAIM OF PRIORITY

This application is related to, and hereby claims the benefit of, U.S. Provisional Patent Application No. 63/022,186, filed May 8, 2020, which is hereby incorporated by reference herein in its entirety.

### BACKGROUND OF THE DISCLOSURE

The present disclosure relates generally to virtual reality systems and more particularly to concurrently rendering an avatar of a patient as an overlay while mirroring the display of a virtual reality headset.

### SUMMARY OF THE DISCLOSURE

Virtual reality (VR) strives to create an immersive virtual world that generates the perception of being physically present in a virtual world. Immersion depends on surrounding the participant with believable images, sounds, and other stimuli. Believable, life-like stimuli elicit a state of consciousness featuring a partial or complete suspension of disbelief that enables action and reaction to stimulations in the virtual environment. An immersive virtual reality system may be a digital hardware and software medical device platform using a combination of virtual environments and full presence tracked avatars for visual feedback.

Virtual reality systems have been used in various applications, including games, and referenced herein may be used in therapeutic regimens to assist patients with their recovery from illness or injury. VR systems may be designed for use in healthcare and focusing on physical rehabilitation and neurorehabilitation. For instance, victims of stroke or brain injury may go to physical therapy for treatment to improve, e.g., range of motion, balance, coordination, joint mobility, flexibility, posture, endurance, and strength. Physical therapy may also help with pain management. Generally, for patients, physical therapy is a vital part of recovery and their goals of performing everyday living functions. VR systems can be used to entertain and instruct patients in their movements while recreating practical exercises that may further therapeutic goals.

Patient movement data during physical therapy sessions may be valuable to both patients and healthcare practitioners. Using sensors in VR implementations of therapy may allow for a deeper immersion as the sensors can capture movements of body parts such as hands and arms and animate a character in the virtual environment. Such an approach may approximate the movements of a patient to a high degree of accuracy. Smarter systems, e.g., with several sensors, may animate movements that mimic the patient more closely, and the patient may better see feedback in virtual activities. Perhaps as important, data from the many sensors may be able to produce statistical feedback for viewing and analysis by doctors and therapists. There exists a need to convert motion of body parts in the real world to a virtual world while preserving the proportionality and accuracy of position and orientation data for real-world data collection and analysis. Moreover, approaches with many sensors may create issues in portability, body sensor placement, and general setup, among other potential problems. For instance, sensors may be placed on a patient's body indiscriminately and upside-down or backwards. There exists a need to simplify sensor placement and/or resolve inverted sensor data in order to reduce human intervention and potential causes of error.

Various systems and methods disclosed herein are described in the context of a therapeutic system for helping patients, such as stroke victims, but this application is only illustrative. In context of the VR system, the word “patient” may be considered equivalent to a user or subject, and the term “therapist” may be considered equivalent to doctor, physical therapist, supervisor, or any non-participating operator of the system. Some disclosed embodiments include a digital hardware and software medical device that uses VR for healthcare, focusing on physical and neuro rehabilitation. The device may be used in a clinical environment under the supervision of a medical professional trained in rehabilitation therapy. In some embodiments, the device may be configured for personal use at home. A therapist or supervisor, if needed, may monitor the experience in the same room or remotely. In some cases, a therapist may be remote or in the same room as the patient. For instance, some embodiments may only need a remote therapist. Some embodiments may require a remote therapist and someone assisting the patient to place or mount the sensors and headset. Generally, the systems are portable and may be readily stored and carried by, e.g., a therapist visiting a patient.

### Overview of an Illustrative Medical Device System According to the Present Disclosure

Disclosed herein is an illustrative medical device system including a virtual reality (VR) system to enable therapy for a patient. Such a VR medical device system may include a headset, sensors, a therapist tablet, among other hardware to enable games and activities to train (or re-train) a patient's body movements.

Virtual reality systems may be used as a part of therapy for patients, for instance, under the supervision of physical therapist or other medical or wellness professionals. VR systems, including, VR headsets, immerse users in an environment, but approaches for therapy should also consider, e.g., multiple patients, therapist goals for each patient, and patient safety.

For instance, some VR system approaches may use virtual reality headset systems similar to those produced for consumer use such as video gaming. Such an approach may not have sufficient durability for several long therapy sessions each day. Some approaches may use large, heavy-duty commercial-based systems that may use, for instance, a tethered headset. Such an approach may not be readily portable or allow for a therapist to transport a VR system to a patient with limited mobility. There exists a need for a durable, portable VR system able to be used in physical therapy sessions.

As described herein, VR systems capable for use in physical therapy may be tailored to be durable, portable and allow for quick and consistent setup. In some embodiments, a virtual reality system for therapy may be a modified commercial VR system using, e.g., a headset and several body sensors configured for wireless communication. A VR system capable of use for therapy may need to collect patient movement data. In some embodiments, sensors, placed on the patient's body, can translate patient body movement to the VR system for animation of a VR avatar. Sensor data may also be used to measure patient movement and determine ranges of motion for patient body parts, e.g., a patient's joints.

### BRIEF DESCRIPTION OF THE DRAWINGS

The above and other objects and advantages of the disclosure will be apparent upon consideration of the following detailed description, taken in conjunction with the accompanying drawings, in which like reference characters refer to like parts throughout, and in which:

FIG. 1A is a diagram of an illustrative system and case, in accordance with some embodiments of the disclosure;

FIG. 1B is a diagram depicting a side view of an illustrative system placed on a participant, in accordance with some embodiments of the disclosure;

FIG. 1C are diagrams depicting front and back views of an illustrative system placed on a participant, in accordance with some embodiments of the disclosure;

FIG. 1D is a diagram of a head-mounted display of an illustrative system, in accordance with some embodiments of the disclosure;

FIG. 2A is a diagram of placing on a participant's head a head-mounted display of an illustrative system, in accordance with some embodiments of the disclosure;

FIG. 2B is a diagram of a sensor and sensor band of an illustrative system, in accordance with some embodiments of the disclosure;

FIG. 2C is a diagram of placing a sensor in a sensor band of an illustrative system, in accordance with some embodiments of the disclosure;

FIG. 2D is a diagram of placing on a participant's hand a sensor and sensor band of an illustrative system, in accordance with some embodiments of the disclosure;

FIG. 2E is a diagram of sensors and sensor bands of an illustrative system, in accordance with some embodiments of the disclosure;

FIG. 2F depicts diagrams of exemplary placement locations for an illustrative system placed on a participant, in accordance with some embodiments of the disclosure;

FIG. 3 is a diagram of an illustrative system, accordance with some embodiments of the disclosure;

FIG. 4 is a diagram of an illustrative system, in accordance with some embodiments of the disclosure;

FIG. 5 is a diagram depicting a side view of an exemplary setup position of a participant using an illustrative system, in accordance with some embodiments of the disclosure;

FIG. 6 is a diagram depicting a side view of an exemplary setup position of a participant using an illustrative system, in accordance with some embodiments of the disclosure;

FIG. 7 depicts an illustrative flowchart of a process for automatic binding of sensors to a patient's body parts, in accordance with some embodiments of the disclosure;

FIG. 8 depicts an illustrative flowchart of a process for automatic binding of sensors to the patient's body parts, in accordance with some embodiments of the disclosure;

FIG. 9 depicts an illustrative flowchart of a process for merging wireless transmitter module (WTM) coordinates into the VR world space, in accordance with some embodiments of the disclosure;

FIG. 10A is an exemplary diagram depicting participant wrist movement and potential range of motion within an illustrative system, in accordance with some embodiments of the disclosure;

FIG. 10B is an exemplary diagram depicting participant wrist movement and potential range of motion within an illustrative system, in accordance with some embodiments of the disclosure;

FIG. 10C is an exemplary diagram depicting participant elbow movement and potential range of motion within an illustrative system, in accordance with some embodiments of the disclosure;

FIG. 10D is an exemplary diagram depicting participant shoulder movement and potential range of motion within an illustrative system, in accordance with some embodiments of the disclosure;

FIG. 10E is an exemplary diagram depicting participant shoulder movement and potential range of motion within an illustrative system, in accordance with some embodiments of the disclosure;

FIG. 11 depicts an illustrative flowchart of a process for automatically correcting sensor orientation, in accordance with some embodiments of the disclosure;

FIG. 12A illustrates a 3D model comprised of a mesh fitted with a skeleton, in accordance with some embodiments of the disclosure;

FIG. 12B illustrates a 3D model comprised of a mesh fitted with a skeleton, in accordance with some embodiments of the disclosure;

FIG. 13 depicts an illustrative flow diagram of a process for creating an avatar mesh, in accordance with some embodiments of the disclosure;

FIG. 14 depicts an illustrative flowchart of a process for creating and scaling an avatar mesh, in accordance with some embodiments of the disclosure;

FIG. 15 depicts an illustrative flowchart of a process for measuring and storing range-of-motion (ROM) data, in accordance with some embodiments of the disclosure;

FIG. 16 depicts an exemplary supervisor interface for an illustrative system, in accordance with some embodiments of the disclosure;

FIG. 17A depicts a chart from an exemplary supervisor interface for an illustrative system, in accordance with some embodiments of the disclosure;

FIG. 17B depicts a chart from an exemplary supervisor interface for an illustrative system, in accordance with some embodiments of the disclosure.

FIG. 18A depicts an exemplary participant interface for an illustrative system, in accordance with some embodiments of the disclosure;

FIG. 18B is a diagram depicting side views of exemplary activity positions of a participant using an illustrative system, in accordance with some embodiments of the disclosure.

### DETAILED DESCRIPTION

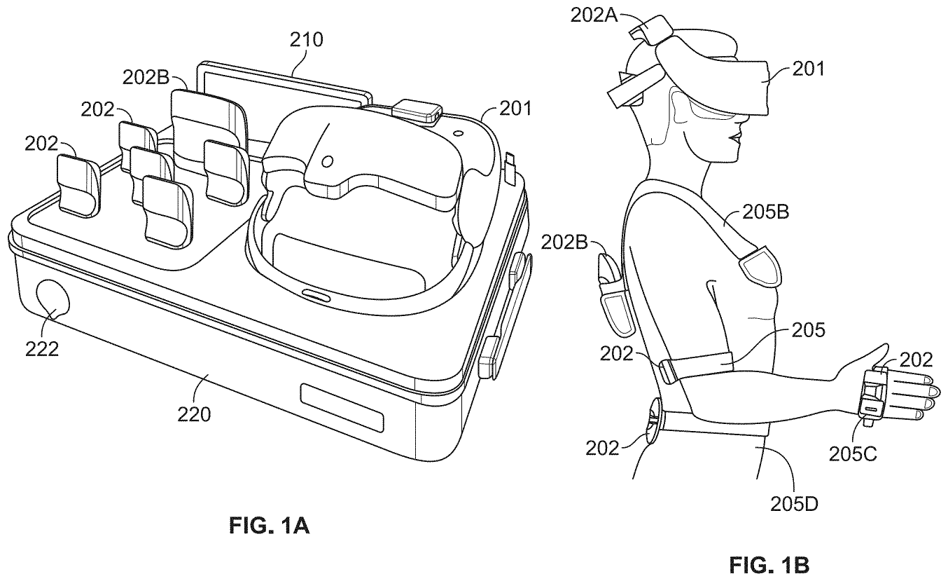

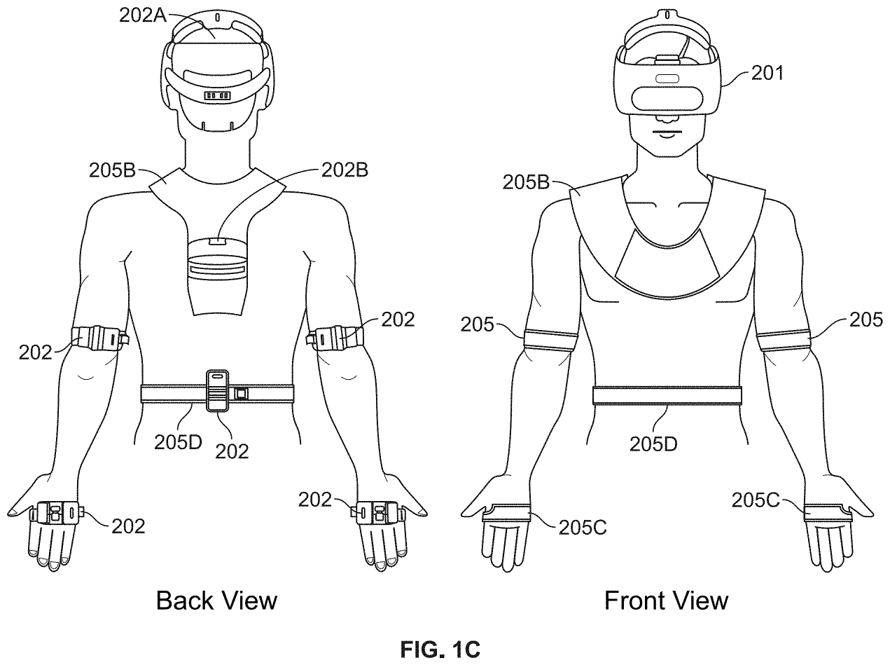

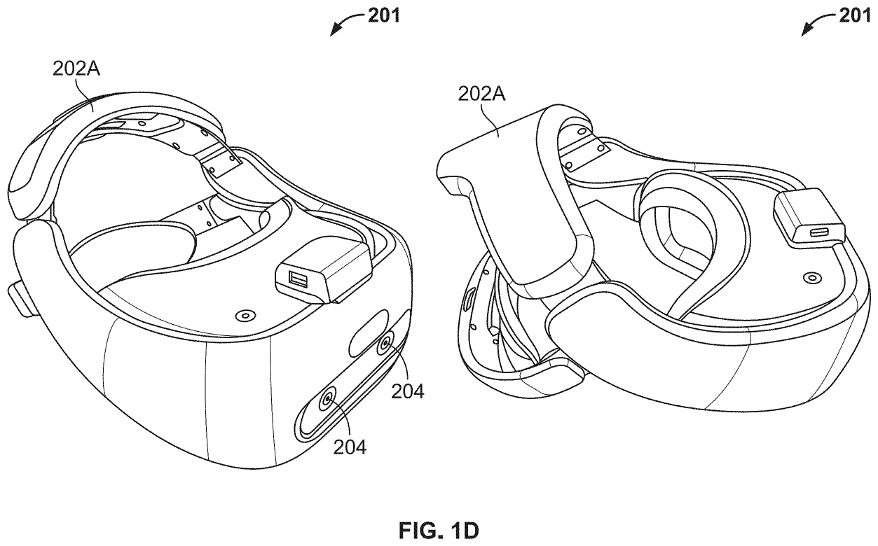

FIG. 1A is a diagram of an illustrative system and case, in accordance with some embodiments of the disclosure. For instance, A VR system may include a clinician tablet 210, head-mounted display (HMD or headset) 201, sensors 202, large sensor 202B, charging dock 220, a router, a router battery, headset controller, power cords, and USB cables.

The clinician tablet 210 may be configured to use a touch screen, a power/lock button that turns the component on or off, and a charger/accessory port, e.g., USB-C. For instance, pressing the power button may power on tablet or restart tablet. Once powered on, a clinician may access a user interface and be able to log in, add or select a patient, initialize and sync sensors, select, start, modify, or end a therapy session, view data, and log out.

A headset 201 may contain a power button that turns the component on or off, as well as a charger/accessory port, e.g., USB-C. The headset also provides visual feedback of virtual reality applications in concert with the clinician tablet and the small and large sensors. HMD 201 may include one or more sensors 202A. HMD 201 may include a wireless receiver, e.g., positioned on or near the headset sensor 202A, configured to receive position and orientation data from sensors 202, 202B transmitted wirelessly via, e.g., 2.4 GHz radio frequency. For instance, each sensor may communicate, via radio frequency, its position and orientation to the HMD receiver every few milliseconds.

Charging headset 201 may be performed by plugging a headset power cord into the storage dock 220 or an outlet. To turn on headset or restart headset, the power button may be pressed. A power button may be on top of the headset. Some embodiments may include a headset controller used to access system settings. For instance, a headset controller may only be used in certain troubleshooting and administrative tasks and not during patient therapy. Buttons on the controller may be used to control power, connect to headset, access settings, or control volume.

The large sensor 202B and small sensors 202 are equipped with mechanical and electrical components that measure position and orientation in physical space and then translate that information to construct a virtual environment. In some embodiments, wearable sensors 202 may be comprised of electromagnetic receivers and emitters, one or more optical elements, infrared emitters, accelerometers, magnetometers, gyroscopes, or a combination thereof. In some embodiments, the processor receives tracking data from both electromagnetic sensors and one or more cameras. In some embodiments, the wearable sensors 202 are wireless and communicate with the HMD and/or other components via radio frequency.

For instance, a VR system may comprise one or more electromagnetic emitters and one or more electromagnetic sensors configured to be selectively placed on one or more tracked body parts. Using processing circuitry in communication with the sensors, emitters, and a visual display such as HMD 201, the processing circuitry is configured to receive tracking data from one or more electromagnetic emitters 202B and the one or more electromagnetic sensors 202, and to generate complementary display data comprising an avatar moving according to sensor data. HMD 201 may include a wireless receiver, positioned on or near the headset sensor 202A, configured to receive position and orientation data from sensors 202, 202B wirelessly via radio frequency. In some embodiments, wireless communications may utilize an integrated low-power RF system-on-a-chip and/or a 2.4-GHz RF protocol stack. For instance, each sensor 202 (and WTM 202B) may communicate, via radio frequency, its position and orientation to the HMD receiver every few milliseconds, e.g., 4 ms.

Sensors are turned off and charged when placed in charging station 220. Sensors turn on and attempt to sync when removed from the charging station. The charging station 220 acts as a dock to store and charge the sensors 202, 202B, tablet 210 and/or headset 201. In some embodiments, sensors 202 may be placed in sensor bands 205 on a patient. Sensor bands 205 may be required for use and are provided separately for each patient for hygienic purposes. In some embodiments, sensors may be miniaturized and may be placed, mounted, fastened, or pasted directly onto the user.

As shown in illustrative FIG. 1A, various systems disclosed herein consist of a set of position and orientation sensors that are worn by a VR participant, in this example, a patient. These sensors communicate with a head-mounted display (HMD) 201, which immerses the patient in a VR experience. An HMD suitable for VR often comprises one or more displays to enable stereoscopic three-dimensional (“3D”) images. Such internal displays are typically high-resolution (e.g., 2880×1600 or better) and offer high refresh rate (e.g., 75 Hz). The displays are configured to present three dimensional images to the patient. VR headsets typically include speakers and microphones for deeper immersion.

An HMD is a central piece to immersing a patient in a virtual world in terms of presentation and movement. A headset may allow, for instance, a wide field of view (e.g., 110°) and tracking along six degrees of freedom. The HMD headset may include cameras, accelerometers, gyroscopes, and proximity sensors. VR headsets typically include a processor, usually in the form of a system on a chip (SoC), and memory. Headsets may also use, for example, additional cameras as safety features to helps users avoid real-world obstacles. An HMD will typically comprise more than one connectivity options in order to communicate with the therapist's tablet. For instance, an HMD may use an SoC that features WiFi, Bluetooth, and/or other radio frequency connectivity, in addition to an available USB connection (e.g., USB Type-C). The USB-C connection may also be used to charge the built-in rechargeable battery for the headset.

The healthcare provider may use a tablet, e.g., tablet 210 depicted in FIG. 1A, to control the patient's experience. The tablet runs an application and communicates with a router to cloud software configured to authenticate users and store information. Tablet 210 may communicate with HMD 201 in order to initiate HMD applications, collect relayed sensor data, and update records on the cloud servers. Tablet 210 may be stored in the portable container and plugged in to charge, e.g., via a USB plug.

FIG. 1B is a diagram depicting a side view of an illustrative system placed on a participant, in accordance with some embodiments of the disclosure. FIG. 1C are diagrams depicting front and back views of an illustrative system placed on a participant, in accordance with some embodiments of the disclosure. In some embodiments, such as depicted in FIGS. 1B-C, sensors 202 are placed on the body in particular places to measure body movement and relay the measurements for translation and animation of a VR avatar. Sensors 202 may be strapped to a body via bands 205. In some embodiments, each patient may have her own set of bands 205 to minimize hygiene issues.

A wireless transmitter module 202B (WTM) may be worn on a sensor band 205B that is laid over the patient's shoulders. WTM 202B sits between the patient's shoulders on their back. In some embodiments, WTM 202B includes a sensor. In some embodiments, WTM 202B transmits its position data in relation to one or more sensors and/or the HMD. In some embodiments, WTM 202B may emit an electromagnetic field (EMF) and sensors 202 are wearable electromagnetic (EM) sensors. For example, wearable sensor 202 may include an EM receiver and a wireless transmitter.

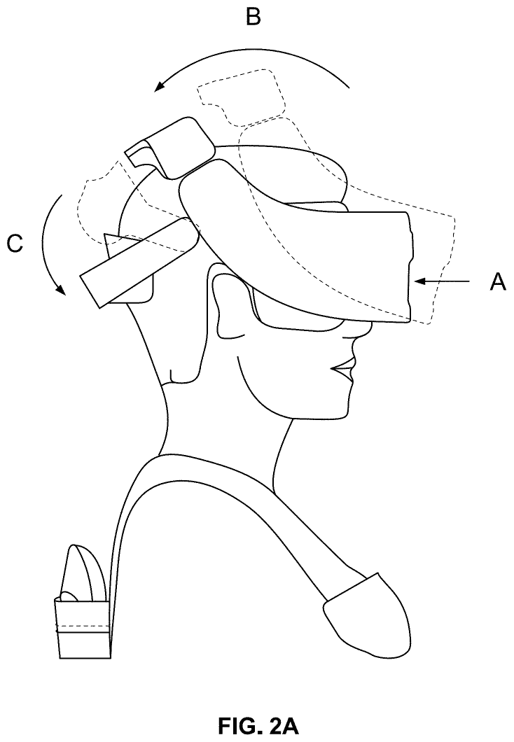

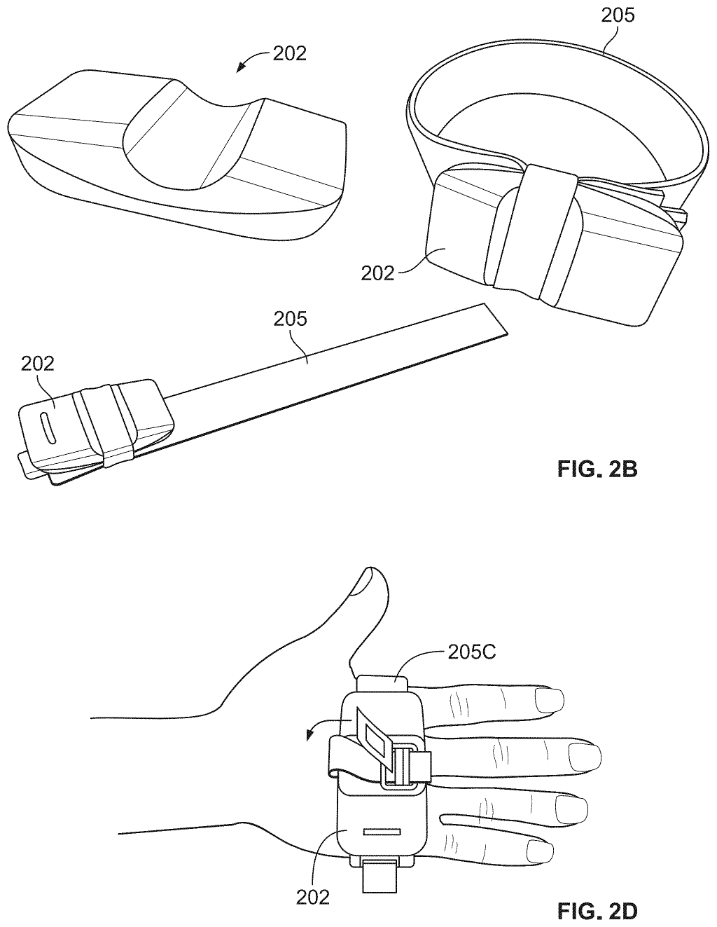

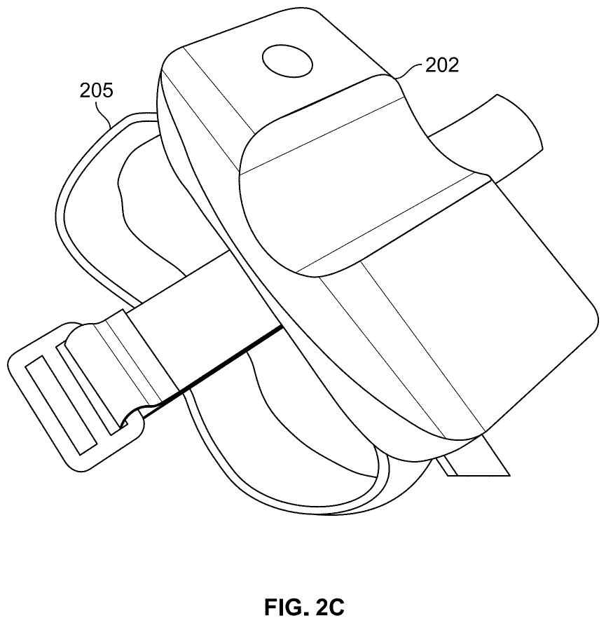

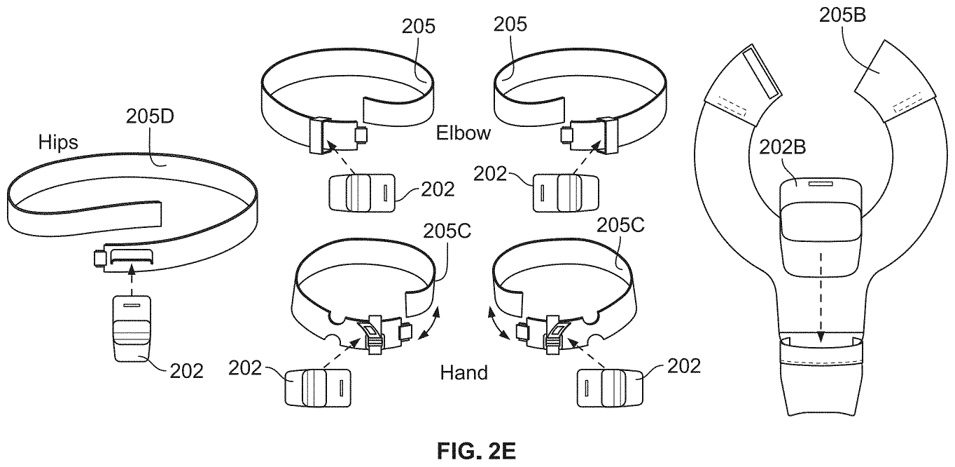

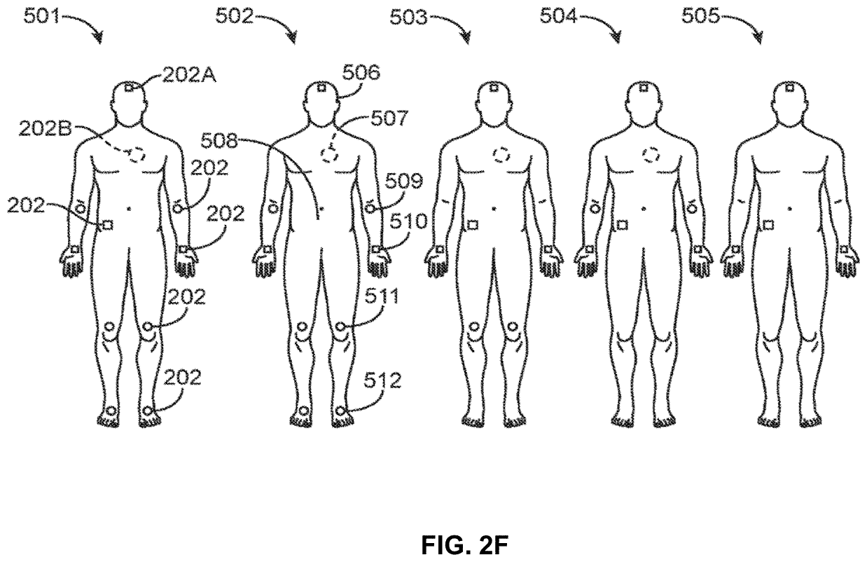

Each sensor 202 may learn its relative position and orientation to the WTM, e.g., via calibration. Sensors 202 with EM receivers in an EMF may provide precise position and orientation tracking with fidelity and precision down to, e.g., the nearest 1 mm (position) and degree (orientation). In some embodiments, wearable sensor 202 may use EMF and inertial measurement. Wireless sensor modules 202 (e.g., sensors or WSMs) are worn just above each elbow, strapped to the back of each hand, and on a pelvis band that positions a sensor adjacent to the patient's sacrum on their back. Wearable sensors 202 may include a light indicating charge status, such as blue or green for charged or charging and red for charge needed. Wearable sensors 202 may be wireless, small, and nonintrusive as illustrated in FIGS. 2A-2E. In some embodiments, each WSM communicates its position and orientation data in real-time to an HMD Accessory (e.g., HMD receiver) located on the HMD.

FIG. 1D is a diagram of a head-mounted display of an illustrative system, in accordance with some embodiments of the disclosure. The HMD Accessory may include a sensor 202A that may allow it to learn its position and orientation relative to WTM 202B. The HMD Accessory may include a wireless receiver which allows the HMD to know where in physical space all the WSMs and WTM are located. The HMD Accessory and receiver may utilize an integrated low-power RF system-on-a-chip and/or a 2.4-GHz RF protocol stack to communicate wirelessly. In some embodiments, each of sensors 202, 202B communicates independently with the HMD Accessory which then transmits its data to the HMD, e.g., via a USB-C connection. In some embodiments, each sensor 202 learns its position and orientation (P&O) based on the EMF emitted by WTM 202B (and other sensor data) and each sensor 202 wirelessly communicates the P&O data with HMD 201, e.g., via radio frequency.

A patient wears HMD 201 on her head and over her face. FIG. 2A is a diagram of placing on a participant's head a head-mounted display of an illustrative system, in accordance with some embodiments of the disclosure. Placement of an HMD on a patient, e.g., depicted in FIG. 2A, may require a therapist to assist. The headset may be adjustable to fit the head comfortably. A headset visor may include an ability to adjust the interpupillary distance between the eyes so that, for example, the displays line-up properly with the eyes in order to produce a three-dimensional view, while maintaining patient comfort. Safety is important for the patient and the therapist. Comfort may be vital for engagement and success of the therapy. The headset may contain a power button that turns the component on or off.

In some embodiments, a first step (A) may include lining up the front of the headset, including, e.g., lining up the visor with the patient's eyes and nose. A second step (B), may include pulling the top of the headset back. In some embodiments, the top of the headset may include a wireless transmitter and sensor 202A. Lastly, in step (C), the back of the headset is placed on the back of the patient's head and adjusted for secure fit and comfort. In some embodiments, HMD 201 may include an adjustment dial to comfortably secure the headset on the patient.

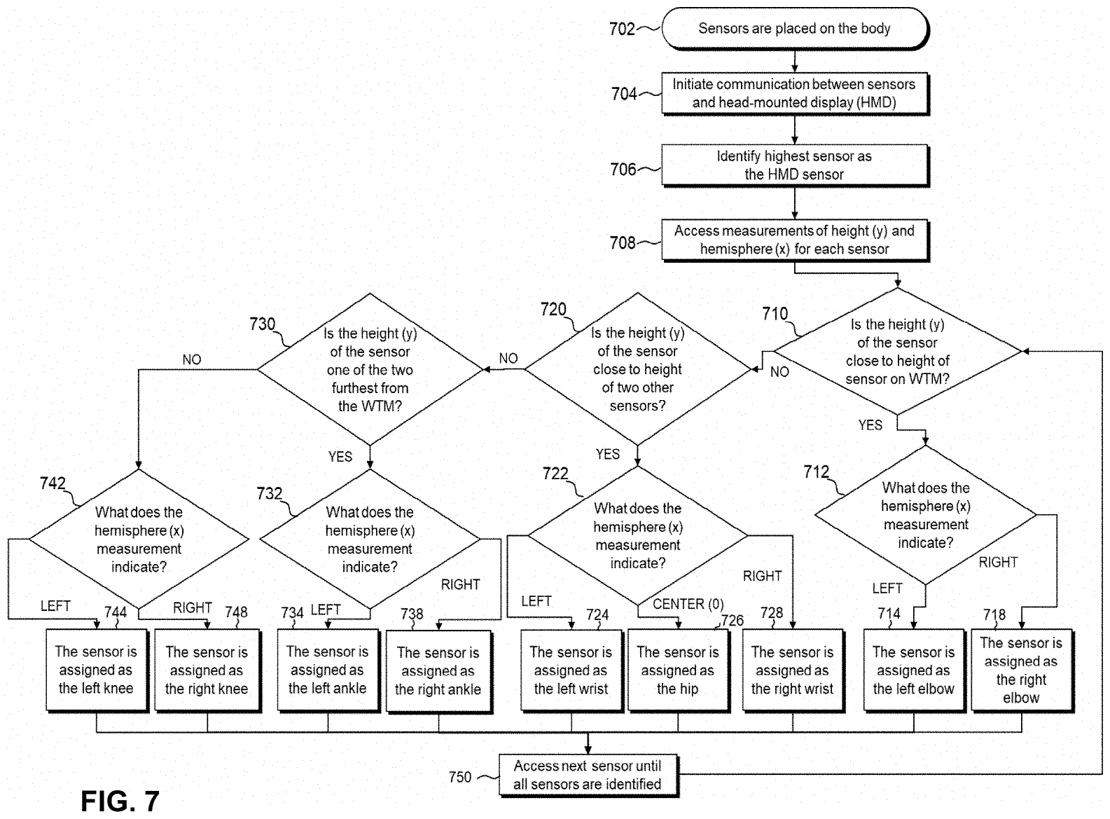

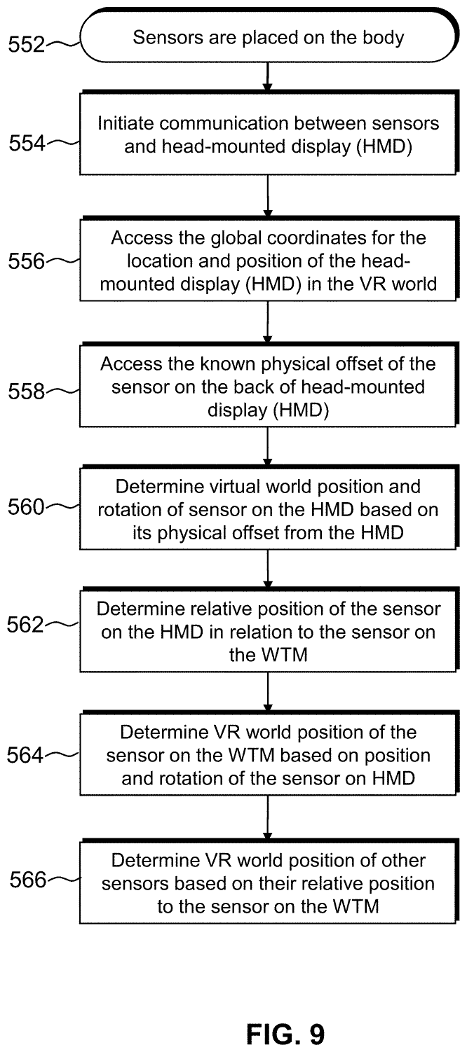

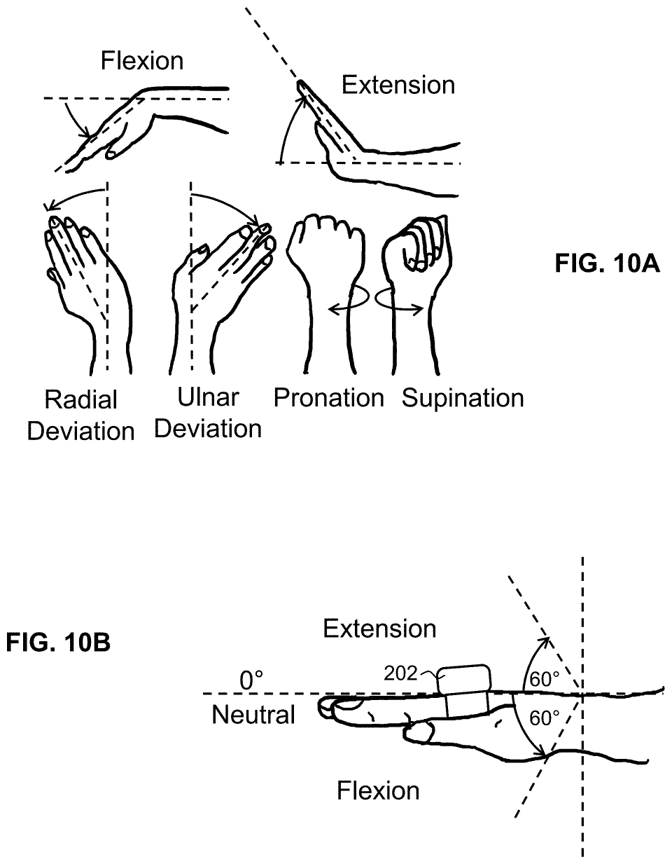

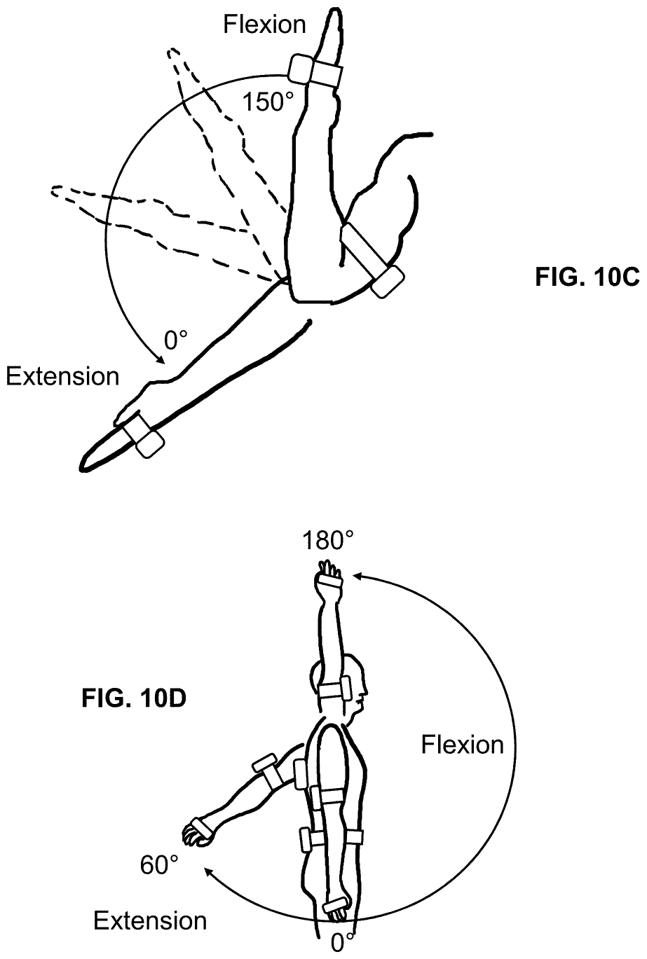

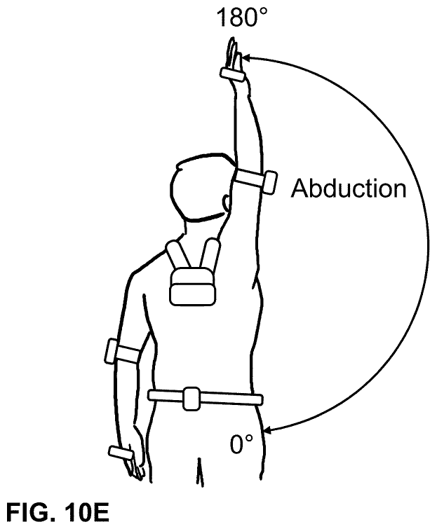

Once the headset is in place and turned on, a patient may begin to explore a virtual reality world. A VR environment rendering engine on the HMD (sometimes referred to herein as a “VR application”), such as the Unreal® Engine, may use the position and orientation data to generate a virtual world including an avatar that mimics the patient's movement and view. Unreal Engine is a software-development environment with a suite of developer tools designed for developers to build real-time 3D video games, virtual and augmented reality graphics, immersive technology simulations, 3D videos, digital interface platforms, and other computer-generated graphics and worlds. A VR application may incorporate the Unreal Engine or another three-dimensional environment developing platform, e.g., sometimes referred to as a VR engine or a game engine. Some embodiments may utilize a VR application, stored and executed by one or more of the processors and memory of a headset, server, tablet and/or other device to render Scenario 100. For instance, a VR application may be incorporated in one or more of head-mounted display 201 and clinician tablet 210 of FIGS. 7A-D and/or the systems of FIGS. 9-10.

A player may “become” their avatar when they log into a virtual reality (“VR”) game. When the player moves their body, they see their avatar move accordingly. Sensors in the headset may allow the patient to move the avatar's head, e.g., even before body sensors are placed on the patient. If a system achieves consistent high-quality tracking, then the player's movements can be completely mapped onto an avatar, which may thereby enable deeper immersion.

An HMD may be essential for immersing a patient in the sights (and head movements) of a VR world, but in order to replicate the real-world movements of body parts below the neck in the VR world, sensors may be used. Sensors may be placed on the body, e.g., of a patient by a therapist, in particular locations to sense and/or translate body movements. The system can use measurements of position and orientation of sensors placed in key places to determine movement of body parts in the real world and translate such movement to the virtual world (and collect data for therapeutic analysis of a patient's range of motion).

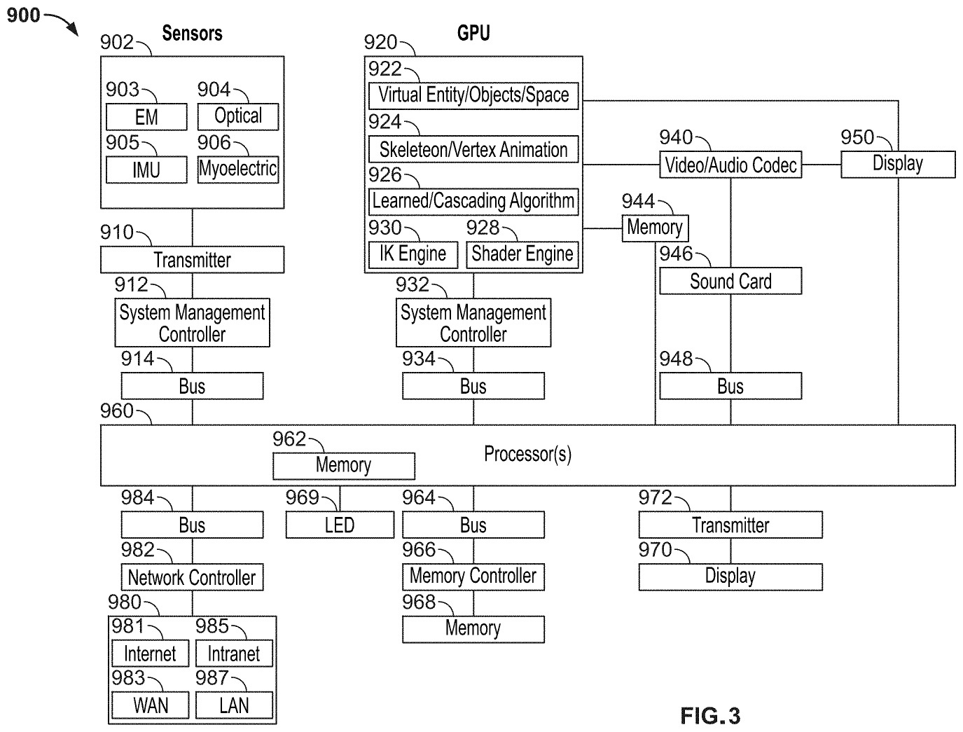

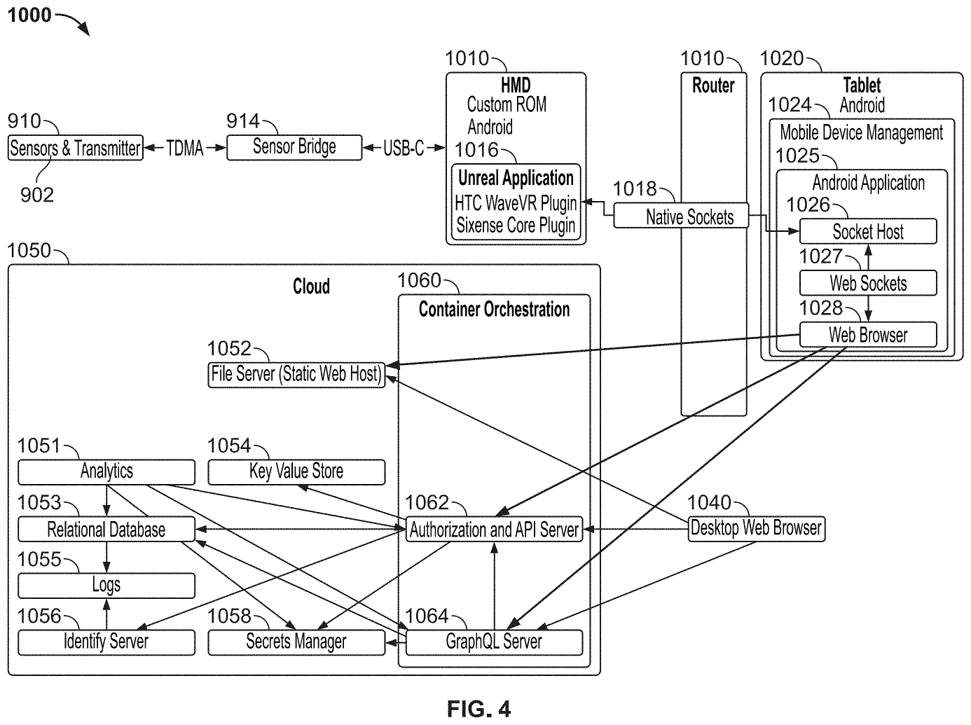

FIG. 2B is a diagram of a sensor and sensor band of an illustrative system, in accordance with some embodiments of the disclosure. FIG. 2C is a diagram of placing a sensor in a sensor band of an illustrative system, in accordance with some embodiments of the disclosure. In some embodiments, systems and methods of the present disclosure may use electromagnetic tracking, optical tracking, infrared tracking, accelerometers, magnetometers, gyroscopes, myoelectric tracking, other tracking techniques, or a combination of one or more of such tracking methods. The tracking systems may be parts of a computing system as disclosed herein. The tracking tools may exist on one or more circuit boards within the VR system (see FIGS. 3-4) where they may monitor one or more users to perform one or more functions such as capture, analyze, and/or track a subject's movement. In some cases, the system may utilize more than one tracking method to improve reliability, accuracy, and precision.

FIGS. 2B-E illustrate examples of wearable sensors 202 and bands 205. To attach sensors 202, the sensors may include a recess that accommodates a cloth and Velcro band 205 that can be used to attach wearable sensors 202 to a subject. In some embodiments, bands 205 may include elastic loops to hold the sensors. This attachment method beneficially does not require the player to hold anything and leaves the hands of the player free during performance of therapeutic exercises. Therapeutic exercises may be performed more easily when a user does not have to hold a controller and the user is not attached by wiring. In some embodiments, bands 205 may include additional loops, buckles and/or Velcro straps to hold the sensors. For instance, bands 205 for hands may require extra secureness as a patient's hands may be moved at a greater speed and could throw or project a sensor in the air if it is not securely fastened. FIG. 2C illustrates an exemplary embodiment with a slide buckle.

FIG. 2E is a diagram of sensors and sensor bands of an illustrative system, in accordance with some embodiments of the disclosure. Sensors 202 may be attached to body parts via band 205. In some embodiments, a therapist attaches sensors 202 to proper areas of a patient's body. For example, a patient may not be physically able to attach band 205 to herself. In some embodiments, each patient may have her own set of bands 205 to minimize hygiene issues. In some embodiments, a therapist may bring a portable case to a patient's room or home for therapy. The sensors may include contact ports for charging each sensor's battery while storing and transporting in the container, e.g., as depicted in FIG. 1A.

As illustrated in FIGS. 2C and 2E, sensors 202 may be placed in bands 205 prior to placement on a patient. In some embodiments, sensors 202 may be placed onto bands 205 by sliding them into the elasticized loops. The large sensor, WTN 202B, is placed into a pocket of shoulder band 205B. Sensors 202 may be placed above the elbows, on the back of the hands, and at the lower back (sacrum). In some embodiments, sensors may be used at the knees and/or ankles. Sensors 202 may be placed, e.g., by a therapist, on a patient while the patient is sitting on a bench (or chair) with his hands on his knees. Sensor band 205D to be used as a hip sensor 202 has a sufficient length to circle a patient's waist.

Once sensors 202 are placed in bands 205, each band may be placed on a body part, e.g., according to placement of sensors depicted in FIG. 1C. In some embodiments, shoulder band 205B may require connection of a hook and loop fastener. An elbow band 205 holding a sensor 202 should sit behind the patient's elbow.

FIG. 2D is a diagram of placing on a participant's hand a sensor and sensor band of an illustrative system, in accordance with some embodiments of the disclosure. In some embodiments, sensor bands 205 may have one or more buckles to, e.g., fasten sensor 202 more securely. For instance, FIG. 2D hand sensor bands 205C features a buckle the facilitate fastening sensor 202 more securely.

Each of sensors 202 may be placed at any of the suitable locations, e.g., as depicted in FIG. 1C. After sensors 202 have been placed on the body they may be assigned or calibrated for each corresponding body part.

Generally, sensor assignment may be based on position of each sensor 202. Sometimes, such as cases where patients have varying height discrepancies, assigning a sensor merely based on height is not practical. In some embodiments, sensor assignment may be based on relative position to, e.g., wireless transmitter module 202B.

FIG. 2F depicts diagrams of exemplary placement locations for an illustrative system placed on a participant, in accordance with some embodiments of the disclosure. FIG. 2F illustrates placement options for potential sensor 202 in accordance with one or more embodiments. In a first example 501, sensors 202 are attached to the head 506, the back 507, the waist 508, the elbows 509, the wrists 510 (or hands), the knees 511, and the ankles 512 for a total of eleven sensors tracking player movement. The sensor placement of this example 501 may be considered optimal for accurately tracking the movements of an entire body. In other embodiments, some, but not all, sensors are attached to a patient. For instance, in a second example 502, the sensors 202 are attached to the head 506, the back 507, the elbows 509, the wrists 510, the knees 511, and the ankles 512 for a total of ten sensors.

In a third example 503, the sensors 202 are attached to the head 506, the back 507, the waist 508, the wrists 510, and the knees 511, for a total of seven sensors. The sensor placement of this example 503 may enable nearly full-body tracking with untracked movements of the elbows and feet being predicted and animated based on the movements of tracked body parts.

In a fourth example 504, the sensors 202 are attached to the head 506, the back 507, the waist 508, the elbows 509, and the wrists 510, for a total of seven sensors. This setup may offer improved tracking of the upper body and is useful for tracking exercises performed while sitting.

In a fifth example 505, the sensors 202 are attached to the head 506, the waist 508, and the wrists 510, for a total of four sensors. This setup may track arm and spine movements well. Typically, sensors are attached to at least the hands/wrists for exercises requiring arm movement, the waist sensor for exercises requiring leaning, and the ankles for exercises requiring leg movement. In any of the forgoing examples, cameras mounted on the player may assist in tracking motion and movements.

FIG. 3 depicts an illustrative arrangement for various elements of a system, e.g., an HMD and sensors of FIGS. 7A-D. The arrangement includes one or more printed circuit boards (PCBs). In general terms, the elements of this arrangement track, model, and display a visual representation of the participant (e.g., a patient avatar) in the VR world by running software including the aforementioned VR application of HMD 201.

The arrangement shown in FIG. 3 includes one or more sensors 902, processors 960, graphic processing units (GPU) 920, video encoder/video codec 940, sound cards 946, transmitter modules 910, network interfaces 980, and light emitting diodes (LED) 960. These components may be housed on a local computing system or may be remote components in wired or wireless connection with a local computing system (e.g., a remote server, a cloud, a mobile device, a connected device, etc.). Connections between components may be facilitated by one or more buses, such as bus 914, bus 934, bus 948, bus 984, and bus 964 (e.g., peripheral component interconnects (PCI) bus, PCI-Express bus, or universal serial bus (USB)). With such buses, the computing environment may be capable of integrating numerous components, numerous PCBs, numerous remote computing systems.

One or more system management controllers, such as system management controller 912 or system management controller 932, may provide data transmission management functions between the buses and the components they integrate. For instance, system management controller 912 provides data transmission management functions between bus 914 and sensors 902. System management controller 932 provides data transmission management functions between bus 934 and GPU 920. Such management controllers may facilitate the arrangements orchestration of these components that may each utilize separate instructions within defined time frames to execute applications. Network interface 980 may include an Ethernet connection or a component that forms a wireless connection, e.g., 802.11b, g, a, or n connection (WiFi), to a local area network (LAN) 987, wide area network (WAN) 983, intranet 985, or internet 981. Network controller 982 provides data transmission management functions between bus 984 and network interface 980.

Processor(s) 960 and GPU 920 may execute a number of instructions, such as machine-readable instructions. The instructions may include instructions for receiving, storing, processing, and transmitting tracking data from various sources, such as electromagnetic (EM) sensors 903, optical sensors 904, infrared (IR) sensors, inertial measurement units (IMUs) sensors 905, and/or myoelectric sensors 906. The tracking data may be communicated to processor(s) 960 by either a wired or wireless communication link, e.g., transmitter 910. Upon receiving tracking data, processor(s) 960 may execute an instruction to permanently or temporarily store the tracking data in memory 962 as, e.g., random access memory (RAM), read only memory (ROM), cache, flash memory, hard disk, or other suitable storage component. Memory 968 may be a separate component in communication with processor(s) 960 or may be integrated into processor(s) 960.

Processor(s) 960 may also execute instructions for constructing an instance of virtual space. The instance may be hosted on an external server and may persist and undergo changes even when a participant is not logged into said instance. In some embodiments, the instance may be participant-specific and the data required to construct it may be stored locally. In such an embodiment, new instance data may be distributed as updates that users download from an external source into local memory. In some exemplary embodiments, the instance of virtual space may include a virtual volume of space, a virtual topography (e.g. ground, mountains, lakes), virtual objects, and virtual characters (e.g., non-player characters “NPCs”). The instance may be constructed and/or rendered in 2D or 3D. The rendering may offer the viewer a first-person or third-person perspective. A first-person perspective may include displaying the virtual world from the eyes of the avatar and allowing the patient to view body movements from the avatar's perspective. A third-person perspective may include displaying the virtual world from, for example, behind the avatar to allow someone to view body movements from a different perspective. The instance may include properties of physics, such as gravity, magnetism, mass, force, velocity, and acceleration, that cause the virtual objects in the virtual space to behave in a manner at least visually similar to the behaviors of real objects in real space.

Processor(s) 960 may execute a program (e.g., the Unreal engine/application discussed above) for analyzing and modeling tracking data. For instance, processor(s) 960 may execute a program that analyzes the tracking data it receives according to algorithms described above, along with other related pertinent mathematical formulas. Such a program may incorporate a graphics processing unit (GPU) 920 that is capable of translating tracking data into 3D models. GPU 920 may utilize shader engine 928, vertex animation 924, and linear blend skinning algorithms. In some instances, processor(s) 960 or a CPU may at least partially assist the GPU in making such calculations. This allows GPU 920 to dedicate more resources to the task of converting 3D scene data to the projected render buffer. GPU 920 may refine the 3D model by using one or more algorithms, such as an algorithm learned on biomechanical movements, a cascading algorithm that converges on a solution by parsing and incrementally considering several sources of tracking data, an inverse kinematics (IK) engine 930, a proportionality algorithm, and other algorithms related to data processing and animation techniques. After GPU 920 constructs a suitable 3D model, processor(s) 960 executes a program to transmit data for the 3D model to another component of the computing environment, or to a peripheral component in communication with computing environment, that is capable of displaying the model, such as display 950.

In some embodiments, GPU 920 transfers the 3D model to a video encoder or a video codec 940 via a bus, which then transfers information representative of the 3D model to a suitable display 950. The 3D model may be representative of a virtual entity that can be displayed in an instance of virtual space, e.g., an avatar. The virtual entity is capable of interacting with the virtual topography, virtual objects, and virtual characters within virtual space. The virtual entity is controlled by a user's movements, as interpreted by sensors 902 communicating with the system. Display 950 may display a Patient View. The patient's real-world movements are reflected by the avatar in the virtual world. The virtual world may be viewed in the headset in 3D and monitored on the tablet in two dimensions. In some embodiments, the VR world is a game that provides feedback and rewards based on the patient's ability to complete activities. Data from the in-world avatar is transmitted from the HMD to the tablet to the cloud, where it is stored for later analysis. An illustrative architectural diagram of such elements in accordance with some embodiments is depicted in FIG. 4.

A VR system may also comprise display 970, which is connected via transmitter 972. Display 970 may be a component of a clinician tablet. For instance, a supervisor or operator, such as a therapist, may securely log into a clinician tablet, coupled to the system, to observe and direct the patient to participate in various activities and adjust the parameters of the activities to best suit the patient's ability level. Display 970 may depict at least one of a Spectator View, Live Avatar View, or Dual Perspective View.

In some embodiments, HMD 201 may be the same as or similar to HMD 1010. In some embodiments, HMD 1010 runs a version of Android that is provided by HTC (e.g., a headset manufacturer) and the VR application is an Unreal application, e.g., Unreal Application 1016, encoded in an Android package (.apk). The .apk comprises a set of custom plugins: WVR, WaveVR, SixenseCore, SixenseLib, and MVICore. The WVR and WaveVR plugins allow the Unreal application to communicate with the VR headset's functionality. The SixenseCore, SixenseLib, and MVICore plugins allow Unreal Application 1016 to communicate with the HMD Accessory and sensors that communicate with the HMD via USB-C. The Unreal Application comprises code that records the Position & Orientation (P&O) data of the hardware sensors and translates that data into a patient avatar that mimics the patient's motion within the VR world. An avatar can be used, for example, to infer and measure the patient's real-world range of motion. The Unreal application of the HMD includes an avatar solver as described, for example, below.

The operator device (in this example, the therapist's tablet) runs a native application (e.g., Android application) that allows an operator such as a physical therapist (PT) to control the patient's experience. The cloud server includes a combination of software that manages authentication, data storage & retrieval, and hosts the user interface which runs on the tablet. This can be accessed by the tablet. The tablet software has several parts.

The first part is a mobile device management 1024 (MDM) layer, configured to control what software runs on the tablet, enables/disables the software remotely, and remotely upgrades the native application.

The second part is a native application, e.g., Android Application 1025, configured to allow an operator to control the HMD Software. The native application, in turn, comprises two parts: (1) a socket host 1026 configured to receive native socket communications from the HMD and translate that content into web sockets, e.g., web sockets 1027, that a web browser can easily interpret; and (2) a web browser 1028, which is what the operator sees on the tablet screen. The web browser receives data from the HMD via the socket host 1026, which translates the HMD's native socket communication 1018 into web sockets 1027, and it receives its UI/UX information from a file server 1052 in cloud 1050. Web browser 1028 may incorporate a real time 3D engine, such as Babylon.js, using a JavaScript library for displaying 3D graphics in web browser 1028 via HTML5. For instance, a real time 3D engine, such as Babylon.] s, may render 3D graphics, e.g., in web browser 1028 on clinician tablet 1020, based on received skeletal data from an avatar solver in the Unreal Engine 1016 stored and executed on HMD 1010.

The cloud software, e.g., cloud 1050, has several different, interconnected parts configured to communicate with the tablet software: Authorization and API Server 1062, GraphQL Server 1064, and File Server (Static Web Host) 1052.

In some embodiments, Authorization and API Server 1062 may be used as a gatekeeper. For example, when an operator attempts to log in to the system, the tablet communicates with the authorization server. This server ensures that interactions (e.g., queries, updates, etc.) are authorized based on session variables such as operator's role, the organization, and the current patient. This server, or group of servers, communicates with several parts of the system: (a) a key value store 1054, which is a clustered session cache that stores and allows quick retrieval of session variables; (b) a GraphQL server 1064, as discussed below, which is used to access the back-end database in order to populate the key value store, and also for some calls to the application programming interface (API); (c) an identity server 1056 for handling the user login process; and (d) a secrets manager 1058 for injecting service passwords (relational database, identity database, identity server, key value store) into the environment in lieu of hard coding.

When the tablet requests data, it will communicate with the GraphQL Server 1064, which will, in turn, communicate with several parts: (1) the authorization and API server 1062; (2) the secrets manager 1058, and (3) a relational database 1053 storing data for the system. Data stored by the relational database 1053 may include, for instance, profile data, session data, game data, and motion data.

In some embodiments, profile data may include information used to identify the patient, such as a name or an alias. Session data may comprise information about the patient's previous sessions, as well as, for example, a “free text” field into which the therapist can input unrestricted text, and a log 1055 of the patient's previous activity. Logs 1055 are typically used for session data and may include, for example, total activity time, e.g., how long the patient was actively engaged with individual activities, activity summary, e.g., a list of which activities the patient performed, and how long they engaged with each on, and settings and results for each activity. Game data may incorporate information about the patient's progression through the game content of the VR world. Motion data may include specific range-of-motion (ROM) data that may be saved about the patient's movement over the course of each activity and session, so that therapists can compare session data to previous sessions' data.

In some embodiments, File Server 1052 serves the tablet software's website as a static web host.

### Use of Sensors in an Illustrative VR System According to the Present Disclosure

The arrangement shown in FIG. 3 includes one or more sensors 902 for measuring, recording, tracking, and transmitting (e.g., via transmitter 910) data in one or more ways. Sensors 902 may include, for instance, electromagnetic (EM) sensors 903, optical sensors 904, infrared (IR) sensors, inertial measurement units (IMUs) sensors 905, and/or myoelectric sensors 906, among other devices and methods. In virtual reality, sensors may be used to collect data on physical movement of the user/patient in order to convert such movement into animation of a VR avatar representing the user. Converting sensor data to motion may also enable control of aspects of the software, such as performing tasks in a game. For instance, in a game where birds are lifted by the patient, the sensor data from the patient lifting her arm may be used to animate the avatar lifting a bird as well as animate the bird moving from one level to another level. This movement data, based on the sensor data, may be stored and displayed, e.g., as angles representing range of motion, to a therapist in a user interface, to allow for more personalized therapy. Sensors, when properly used, may provide visual feedback, virtual world task feedback, and therapist feedback, among other types of feedback.

Sensor hardware is used to measure positions and orientations of body parts in order for a VR system to translate a patient's movements into VR avatar animations. In some approaches, sensors may be tailored to particular body parts and have specific functions and measurements. For instance, hands/wrist sensors may be different than elbow sensors, which may be different than a hip sensor. In some cases, sensors may be custom made for left or right sides. Such an approach may be time consuming and expensive to manufacture. Such an approach may be time consuming and expensive to setup on a patient. Such approaches may be compatible with certain features of VR systems described herein, however, there exists a need for a VR system with same sensors and able to be assigned to various body parts.

In some embodiments described herein a sensor may be suitable for any position in the VR sensory system. For example, the hardware of a sensor used on the hips may be identical to the hardware used on a left elbow.

In some embodiments, electromagnetic tracking may be enabled by running alternating current through one or more ferrite cores with three orthogonal (x, y, z) coils, thereby transmitting three dipole fields at three orthogonal frequencies. The alternating current generates a dipole, continuous wave electromagnetic field. With multiple ferrite cores, differentiation between cores may be achieved using frequency division multiplexing. U.S. Pat. Nos. 8,520,010 and 10,162,177 provide additional details and are hereby incorporated by reference herein in their entireties. The cores may function to emit and/or receive EM signals from each other, ferrous objects around the user, and/or the earth's magnetic field to determine the position and orientation of the core and thus the sensor.

In some embodiments, for instance, a WTM

Tracking may be further enabled by inertial measurement units (IMUs). IMUs may include accelerometers, magnetometers, and gyroscopes. Accelerometers measure the rate of change of the velocity of a given PCB undergoing movement in physical space. Magnetometers characterize magnetic field vectors by strength and direction at a given location and orientation. Gyroscopes utilize conservation of angular momentum to determine rotations of a given PCB. The individual components of an IMU serve to supplement, verify, and improve the tracking data captured by electromagnetic sensors. In one example, the wearable sensors 202 depicted in FIGS. 2A-C utilize a combination of electromagnetic tracking and IMU tracking to capture, analyze, and track a user's movements.

Optical tracking and infrared tracking may be achieved with one or more capture devices. In some embodiments, the system may perform tracking functions using a combination of electromagnetic tracking and optical tracking. In some cases, a camera is worn by the user. In some cases, capture devices may employ an RGB camera, time-of-flight analysis, structured light analysis, stereo image analysis, or similar techniques. In one example of time-of-flight, the capture device emits infrared (IR) light and detects scattered and reflected IR light. By using pulsed IR light, the time-of-flight between emission and capture for each individual photon indicates the distance the photon traveled and hence the physical distance of the object being imaged. This may allow the camera to analyze the depth of an image to help identify objects and their locations in the environment. Similar techniques may analyze reflected light for phase shifts, intensity, and light pattern distortion (such as bit maps). Stereo image analysis utilizes two or more cameras separated by some distance to view a similar area in space. Such stereo cameras capture a given object at one or more angles, which enables an analysis of the object's depth. In one example, as depicted in FIG. 4A, the HMD 201 may utilize one or more cameras 204 that enable optical tracking to identify an object or location in physical space to serve as an anchor, e.g., (0, 0, 0). The tracking system may then determine global movements in reference to the anchor.

Myoelectric tracking may be achieved using multiple sensors capable of sensing nerve impulse (EMG) signals. The sensors may be attached with a band, with leads, or with a needle electrode. The EMG signals being decoded into a model of intended movements by a learned algorithm executed, at least, in part by a processor as discussed below. Monitoring EMG activity can be useful for measuring the neural activity associated with neuroplasticity.

In one specific example, the electromagnetic sensors each include a receiver (RX) module having three orthogonal coils that are configured to receive an electromagnetic field generated by a transmitter (TX), which also includes three orthogonal coils. The magnetic field data collected at each coil is processed by a Discrete Fourier Transformation (DFT). With three coils on each module, the signal received by a module is representable by a 3×3 signal matrix (“Sigmat”), which is a function of a transmitter-to-sensor radius vector and a transmitter-to-sensor rotation matrix (e.g., directional cosines or projection matrix). An IMU and camera system may be used to correct for errors in electromagnetic tracking. In one example, a dipole field approximation allows for the determination of position and orientation (P&O) according to Equation 1, as described in U.S. Pat. No. 4,737,794 (which is hereby incorporated by reference herein in its entirety).

X=N^{T}B(r) Equation No. 1:

X—three-by-three (3×3) Sigmat Matrix (as sensed in RX coordinates)

N—three-by-three (3×3) orthonormal orientation (in TX coordinates) Transmitter to sensor rotation matrix (6 values received from IMUs), and where T notation designates the transpose of a matrix.

r—three-by-one (3×1) position vector (in TX coordinates) (transmitter to sensor radius vector)

B—three (3) magnetic fields at r as the columns of a 3×3 matrix (in TX coordinates)

Distortion and interference may be compensated for by adding E(r) to the equation. E(r)is a result calculated from the super position of the theoretic dipole fields and is represented as a 3×3 matrix of unknown magnetic field distortion or interference. E(r) may be described as an error matrix in that it, e.g., compensates for errors in calculated P&O, as described in U.S. Pat. No. 9,459,124.

X=N^{T}(B(r)+E(r)) Equation No. 2:

E(r) may be calculated using data from IMUs and a camera system (as explained in more detail below). Each IMU typically includes an accelerometer, a gyroscope, and a magnetometer. These components help correct for error, noise, and phase ambiguity in P&O calculations, as described in U.S. Pat. No. 10,234,306, which is hereby incorporated by reference herein in its entirety. For example, assume Sigmat is being distorted by a nearly uniform EM field generated by a large wire loop on the floor. To model distortion, the direction of the distortion field (v) and the gains per frequency (P) may be determined.

E(r)=v·P The Distortion field:

v—three-by-one (3×1) direction of the distortion field (same for all three frequencies)

P—one-by-three (1×3) gains for the distortion field per frequency (scalar)

X=N^{T}(B(r)+v·P) Equation No. 3:

Position and orientation (P&O) may also be corrected by a gravity equation derived from a fusion of the IMU's accelerometer and gyroscope by means of a Kalman filter sensor fusion, as detailed in US Patent Application No. 2016/0377451A1, which is hereby incorporated by reference herein in its entirety.

N·Gᵣₓ=Gₜₓ Gravity equation:

A portion of the gravity equation can be substituted for direction of the distortion field (v). This substitution simplifies the distortion field to the roll about gravity, which reduces the number of unknown variables and makes the equation more easily solved. The equation is easier to solve because it reduces the degrees of freedom (DOF) of N (orientation) from 3 angles to just 1 (roll about gravity). U.S. Pat. No. 10,162,177 may describe this in more detail and with more information. Substituting the direction of the distortion field v in Equation No. 3 with Gᵣₓ yields Equation No. 4:

X=N^{T}B(r)+Gᵣₓ·P Equation 4:

Accordingly, seven parameters must be determined in order to solve Equation No. 4:

θ—roll angle of N

r—three dimensional (3D) position vector

P—distortion gains

The Sigmat has 9 values, and 9 is greater than 7, so a unique solution is probable. Solving the equation analytically is difficult, however iterative optimization methods offer a simpler solution through the use of a Jacobian. (e.g. Levenberg-Marquardt algorithm).

F(θ,r,P)=∥N(θ)^{T}B(r)+Gᵣₓ·P−X∥² Equation No. 5 (SOLVER 1):

First, (θ, r) are initialized using an analytic dipole solution (ignoring distortion) or by tracking, initialize P=(0,0,0). Next, the Jacobian of F(θ, r, P) is computed using numerical derivatives. The Jacobian is used to compute a step which decreases F. A final calculation step is to perform iterations until some tolerance is achieved. The value of corrected P&O is then compared to measured P&O to determine the ratio of unexplained Sigmat and confidence intervals. Equation No. 6 is used for blending the three solvers.

$${E}_{x} = \frac{‖ {X}_{P \& O} - {X}_{Measured} ‖}{‖ {X}_{Measured} ‖} \qquad Equation 6$$

When EM+IMU fusion provides the constraint, the equation becomes:

X=N^{T}B(r)+v·P Equation No. 7 (SOLVER 2):

Where N=N_{fusion}

### Merging of Electromagnetic and Optical Coordinate Systems According to the Present Disclosure

In some embodiments, the electromagnetic tracking system is self-referential, where P&O is established relative to a wearable transmitter with unknown global coordinates. A self-referential tracking system can be merged with a global coordinates system in many ways. In one example, embodiments of the present disclosure provide a system including a camera 204 as depicted in FIG. 1D. The camera 204 records and analyzes images of the patient's surroundings to establish an anchor (e.g., (0, 0, 0)). The movement of this camera 204 is calculated as movements relative to this global coordinate anchor point.

Some embodiments disclosed herein include a sensor, e.g., sensor 202A, configured to enable the tracking system's translation from self-referential coordinates to global coordinates. Such a sensor 202A has a fixed position relative to the system's cameras 204. This fixed position provides a known distance and orientation between the self-referential coordinates and the global coordinate, allowing their merger, as described in U.S. Pat. No. 10,162,177.

When merged, the benefits of both coordinate systems are maximized while the downsides are minimized. Anchoring a tracking system in real space and accurately positioning the patient, as a whole, in VR may be best achieved by an optical system. However, an optical system is limited by line of sight and is therefore not ideal for determining player positional nuances, such as limb location and other body configuration information. On the other hand, an electromagnetic system is excellent at tracking limb position and body configuration, but typically requires a stationary transmitter for position tracking relative to a real-world reference. By combining the two systems, the entire system of sensors may be optimized to be both mobile and accurate.

### Automatic Assignment of Sensors to a Wearer's Body Parts According to the Present Disclosure

As disclosed herein, sensors may be configured to be interchangeably placed on a patient's body and function properly after an automatic assignment of each sensor to its corresponding location on the body.

In a virtual reality system, if each sensor is not associated with the proper body location for the session, sensor data may be incorrect. If sensor data is incorrect, the animation will reflect an error, the VR activity control will not function correctly, and the therapist's patient data will be unsuitable for comparison. Accordingly, such sensor association should not be left open to opportunities for error.

A system needs to identify, for instance, if the measurements reflect movement of an elbow or a wrist. A system needs to also identify, for instance, whether a movement occurs on the patient's left side or right side. Sensor data collection, like any medical measurement, should be made as precisely and accurately as possible. Errors in sensor data collection, such as associating a sensor with the wrong body part, could be detrimental to a patient's therapy.

If a sensor mistake is made, data could be recorded in in an improper field. For instance, if sensor data were recorded for the incorrect side of the body, the data and results could sabotage the patient's progress records. Improper data records could potentially cause misdiagnosis and/or improper resulting treatment. With incorrect data, a therapist might introduce activities or exercises that could cause the patient to experience pain, or otherwise harm the patient. Misidentifying sensors and/or improper sensor placement could lead to improper patient care, as well as potential patient injury.

In some approaches, systems may have particular sensors for each body part to ensure data is collected for the proper body part. For instance, each sensor might have a particular shape. Such an approach may invite human error in placing the sensors, which could lead to mistakes, inappropriate activity, and patient injury. In order to minimize risk of mistake, a physical therapist may have to spend additional time installing each sensor on the appropriate body part. Additional time may cost money and/or may lead to patient issues such as loss of focus.

Each sensor may have a label. Such an approach may be confusing and enable user error in placing the sensors with, e.g., straps. For instance, even with labels, sensor setup may require additional time and attention. A “left” label indicating a sensor for a patient's left upper-arm could be mistaken for a therapist's left side, which could lead to a swap of sides. A virtual avatar reacting to Mistaking the left arm sensors for the right arm sensors could produce poor data which could lead to potentially injurious patient activities on a less strong or developed arm. Such patient injuries may be detrimental to therapy.

In some approaches, systems may have identical sensors but each sensor is preassigned to a body part. Such a system may use physical labels for the sensors to ensure they are placed in the correct position. Sensors that are not immediately recognizable for each body part may have potential issues of placement delay, swapped hemispheres, difficult replaceability and other human error. There exists a need for interchangeable, swappable sensors.

As disclosed herein, interchangeable sensors provide a solution that reduces placement errors and minimizes setup time. In some embodiments, interchangeability of sensors may facilitate storage. For example, storing interchangeable sensors in charging docks within a portable container may allow quicker packing and retrieval. When packing away sensors, a therapist will not have to put the interchangeable sensors in specific or labeled docks. Certain sensor systems with swappable sensors may allow therapists to replace inoperative sensors quickly. For instance, if a sensor is broken or has an uncharged/low battery, a new sensor may be swapped into the system.

In some embodiments, sensors may be interchangeably positioned on the body. Interchangeability may allow easier setup and quicker storage and mobility. For instance, the system may be portable and allow the therapist to visit multiple patients in different rooms of a medical building or, potentially, at their homes. In some embodiments, sensors may be stored in a charging unit, e.g., a mobile dock. FIG. 1A depicts a portable storage container. A mobile charging unit may have docks for each sensor. When the sensors are removed from their docks, the interchangeable sensors may be placed on the body in any order.

Sensors may be attached by elastic bands, such as bands 205 depicted in FIGS. 2B-E. As illustrated in FIG. 2C, sensors 202 may be placed in bands 205 prior to placement on a patient. In some embodiments, sensors 202 may be placed onto bands 205 by sliding them into the elasticized loops. Sensors, for instance, may be placed above the elbows, on the back of the hands, and at the lower back (sacrum). In some cases, sensors may be placed on bands already attached to a patient, but sensors are typically placed in the bands first to ensure proper comfort and safety in securing the sensors.

If the sensors are interchangeable, then the system must be able to assign each sensor to the proper part. Some approaches may require manual assignment, which would diminish any time efficiency from having interchangeability. There exists a need to automatically assign each sensor to the proper body part within the VR system.

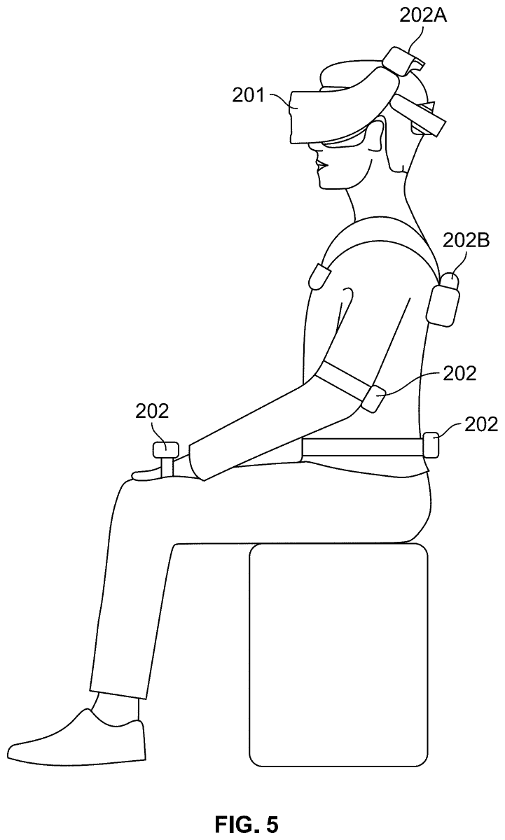

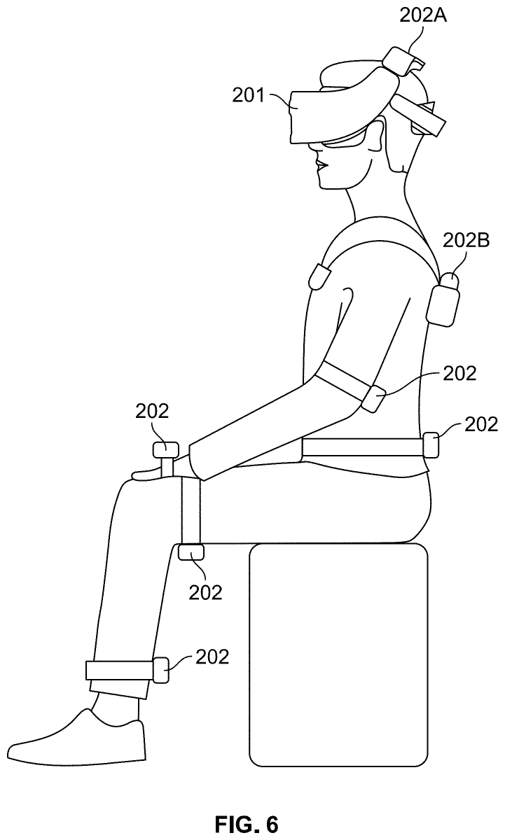

As disclosed herein, interchangeable sensors can be automatically assigned to any body part during a calibration or syncing mode when the sensors are first placed on each patient. In some embodiments, each of the wearable sensors are initially unassigned and, upon startup and placement, the sensors may begin to auto-sync. Automatic Binding may allow for seamless, reduced-error setup, and requires minimal or no manual input. Once the sensors are placed on the body, the system automatically determines where on the body each sensor has been placed and assigns them as such. This automatic binding feature improves on ease of use by simplifying and expediting the process of starting the system. In some embodiments, LEDs on the sensors may indicate the sensor is syncing or has been properly synced. Some illustrative arrangement of sensors are shown below in FIG. 5 and FIG. 6 during a potential position for setup. Some embodiments of sensor placement are also depicted in other figures, such as FIGS. 1C and 2F.

In some embodiments, upon startup and placement, sensors 202 will begin to auto-sync Each of the wearable sensors 202 are initially unassigned. Once the sensors 202 are placed on the body, the system may automatically determine where on the body each sensor has been placed and assigns them as such. In one example, the sensors placed on the body provide position and orientation (P&O) data relative to a sensor with an emitter worn on a user's back. The P&O data may then be analyzed by the host to determine the positioning of the various sensors. At least two variables may be used to determine the location of every sensor, height, and hemisphere (e.g., right or left side). The sensor with the highest position is easily identified as the sensor on the HMD. Sensors 202 having a height closest to the emitter sensor are assigned as the left and right elbows, respectively. Moving down on the subject, three sensors 202 may be detected at positions at about waist-height. A center-most sensor 202 at this height is assigned as the waist sensor, and the left sensor is assigned as the left hand and the right sensor is assigned as the right hand. In some embodiments knee and ankle sensors are similarly identified by their hemisphere (left or right) and their height. Although the variable height and hemisphere were used in the example above, this should be understood as a general process to achieve auto-syncing. For instance, the magnetic field vectors received at each sensor must be processed before height and hemisphere can be determined. The magnetic field vectors may alternatively be processed to determine absolute distance from an emitter. In some embodiments, if the player moves his or her arms, accelerometers inside the sensors may help identify the hand/wrist and elbow sensors. During such arm movements, typically the hands/wrists will have the greatest acceleration of all the sensors, and the elbows will an acceleration lower than the hands/wrists and higher than the other sensors 202. The rest of the sensors 202 may then be determined by height. The present invention may use other such processing methods, as known by those with skill in the art, or combinations of such methods, to determine relative sensor location. Auto-body-positioning may allow for seamless, error-proof setup, and requires no manual input. This auto-syncing feature may improve ease of use by simplifying and expediting the process of starting the system, so physical therapy can be started quickly.

FIG. 5 is a diagram depicting a side view of an exemplary setup position of a participant using an illustrative system, in accordance with some embodiments of the disclosure. In FIG. 5, for example, sensors 202 as depicted in FIG. 5 placed on the body provide P&O data, e.g., relative to a sensor 202B worn on a user's back (e.g., in a wireless transmitter module (WTM)). The P&O data is then analyzed by the host to determine the positioning of the various sensors. Two variables can be used to determine the location of every sensor, height and hemisphere (e.g., right or left side).

FIG. 6 is a diagram depicting a side view of an exemplary setup position of a participant using an illustrative system, in accordance with some embodiments of the disclosure. In FIG. 6, for example, the sensor with the highest position is identified as the sensor 202A on the HMD 201. The sensors 202 having a height closest to the emitter sensor 202B worn on the back are assigned as the left and right elbows, respectively. Moving down, three sensors 202 are positioned at about waist height. A middle-most sensor at this height is assigned as the waist sensor, and the left sensor is assigned as the left hand/wrist and the right sensor is assigned as the right hand/wrist. The knee and ankle sensors may be similarly identified by their hemisphere (left or right) and their height.

FIG. 7 depicts an illustrative flowchart of a process for automatic binding of sensors to a patient's body parts, in accordance with some embodiments of the disclosure. Some embodiments may utilize an avatar engine, e.g., as part of a VR application, stored and executed by one or more of the processors and memory of a headset, server, tablet, and/or other device.

The process begins after the sensors are placed on the body at step 702. In some embodiments, sensors may be applied to, for instance, the patient's head, back, hips, elbows, hands/wrists, knees, and ankles. In the process of FIG. 7, the patient is sitting with her hands on her knees. In this process, a wireless transmitter module (WTM) includes a sensor and is worn on a sensor band that is laid over the patient's shoulders. In this process, the head-mounted display (HMD) includes a sensor, as well.

At step 704, the avatar engine initiates communication between sensors and the HMD. In some embodiments, each WSM wirelessly communicates its position and orientation with the HMD receiver. In some embodiments, the WTM sensor wirelessly communicates with the HMD receiver. In the examples in FIGS. 1B-C, 5, and 6, the sensor attached to the WTM on the patient's back is identified as sensor 202B and depicted with the other sensors 202.

At step 706, the avatar engine identifies the highest sensor as the sensor on the head-mounted display. In the example of the patient in FIG. 6, the sensor with the highest position is identified as the sensor 202A on the HMD 201.

At step 708, the avatar engine accesses measurements of height (y) and hemisphere (x) for each sensor. In some embodiments, hemisphere may be measured as, e.g., right or left side. In some embodiments, hemisphere may be a measurement of horizontal distance.

At step 710, the avatar engine determines if the height (y) of the sensor close to height of sensor on WTM. If the height (y) of the sensor is close to height of sensor on WTM, then the sensor is most likely one of two sensors at the elbow. Then, at step 712, the avatar engine determines which hemisphere (x) the measurement indicates. If the sensor's hemisphere (x) measurement indicates left then, in step 714, the sensor is assigned as the left elbow. If the sensor's hemisphere (x) measurement indicates right then, in step 718, the sensor is assigned as the right elbow. After assignment of the sensor, the next sensor is accessed, at step 750, until all sensors are identified.

If the avatar engine determines, at step 710, that the height (y) of the sensor is not close to height of sensor on WTM, then the avatar engine determines, at step 720, if the height (y) of the sensor is close to height of two other sensors. The three sensors that are similar heights are the hands/wrist sensors and the hip sensor. Then, at step 722, the avatar engine determines which hemisphere (x) the measurement indicates. If the sensor's hemisphere (x) measurement indicates left then, in step 724, the sensor is assigned as the left hand/wrist. If the sensor's hemisphere (x) measurement indicates center (e.g., close to 0) then, in step 726, the sensor is assigned as the hip. If the sensor's hemisphere (x) measurement indicates right then, in step 728, the sensor is assigned as the right hand/wrist. After assignment of the sensor, the next sensor is accessed, at step 750, until all sensors are identified.

If the avatar engine determines, at step 720, that the height (y) of the sensor is close to height of two other sensors, then the avatar engine determines, at step 730, the avatar engine determines if the height (y) of the sensor is the furthest (pair) from the sensor on WTM. If the height (y) of the sensor is the furthest (pair) from the sensor on WTM, then the sensor is most likely one of two sensors at the ankles. Then, at step 732, the avatar engine determines which hemisphere (x) the measurement indicates. If the sensor's hemisphere (x) measurement indicates left then, in step 734, the sensor is assigned as the left ankle. If the sensor's hemisphere (x) measurement indicates right then, in step 738, the sensor is assigned as the right ankle. After assignment of the sensor, the next sensor is accessed, at step 750, until all sensors are identified.

If the avatar engine determines, at step 720, that the height (y) of the sensor is not the furthest (pair) from the sensor on WTM, then the sensor is most likely one of two sensors at the knees. Then, at step 742, the avatar engine determines which hemisphere (x) the measurement indicates. If the sensor's hemisphere (x) measurement indicates left then, in step 744, the sensor is assigned as the left knee. If the sensor's hemisphere (x) measurement indicates right then, in step 748, the sensor is assigned as the right knee. After assignment of the sensor, the next sensor is accessed, at step 750, until all sensors are identified.

At step 750, when the next sensor is accessed, until all sensors are identified, the process loops back to step 710 and the height is examined.

Although the variable height and hemisphere were used in the example above, this should be understood as a simplification of one way to achieve auto-syncing. For instance, the magnetic field vectors received at each sensor must be processed before they can determine height and hemisphere. The magnetic field vectors may alternatively be processed to determine absolute distance from an emitter. Additionally, if the participant moves his or her arms, accelerometers inside the sensors may help identify the hand/wrist and elbow sensors. During arm movements, typically the hands/wrists will have the greatest acceleration of all the sensors, and the elbows may an acceleration lower than the wrists and higher than the other sensors. The rest of the sensors may then be determined by height alone. Systems of the present disclosure may use other such processing methods or combinations of such methods, to determine relative sensor location.

In another example, sensors may be placed on a wearer's hands, elbows, and pelvis. Automatic binding begins with the wearer sitting still, hands resting on their thighs. The distance of the various sensor relative to the HMD are checked to ensure they fall within a predefined distance. Sensor movement may also be determined to ensure they are still, or near still. The pelvis sensor is first identified by identifying which of the unassigned sensors is closest to the WTM on the X and Y axes. The system then evaluates if this sensor is physically located below the WTM, and if the angle of the sensor is mostly aligned upright against the patient's spine. If all requirements are met, this sensor is assigned as the pelvis sensor.

The system next determines which two remaining sensors are most likely to represent the left and right hands. Assuming the patient has their hands resting on their thighs, the system determines which two sensors are furthest away from the WTM's Y plane in the forward direction. Each of these two sensors are measured relative to the WTM's X plane. If a sensor has a negative X value, it is considered to be the right hand. A positive X value means the sensor is the left hand.

The two remaining sensors (excluding the sensor that is directly attached to the HMD) are then bound as the elbow sensors. Similar to the hands, the system determines the X coordinates of these sensors relative to the WTM. If the X value is negative, the sensor represents the right elbow. If the X value is positive, the sensor is assigned as the left elbow.

In some embodiments, the system makes a final check to ensure that the left hand and left elbow sensors are on the left side of the virtual avatar and that the right hand and right elbow sensors are on the right side. If one isn't on the correct side of the body, it is corrected before the virtual reality avatar is rendered.

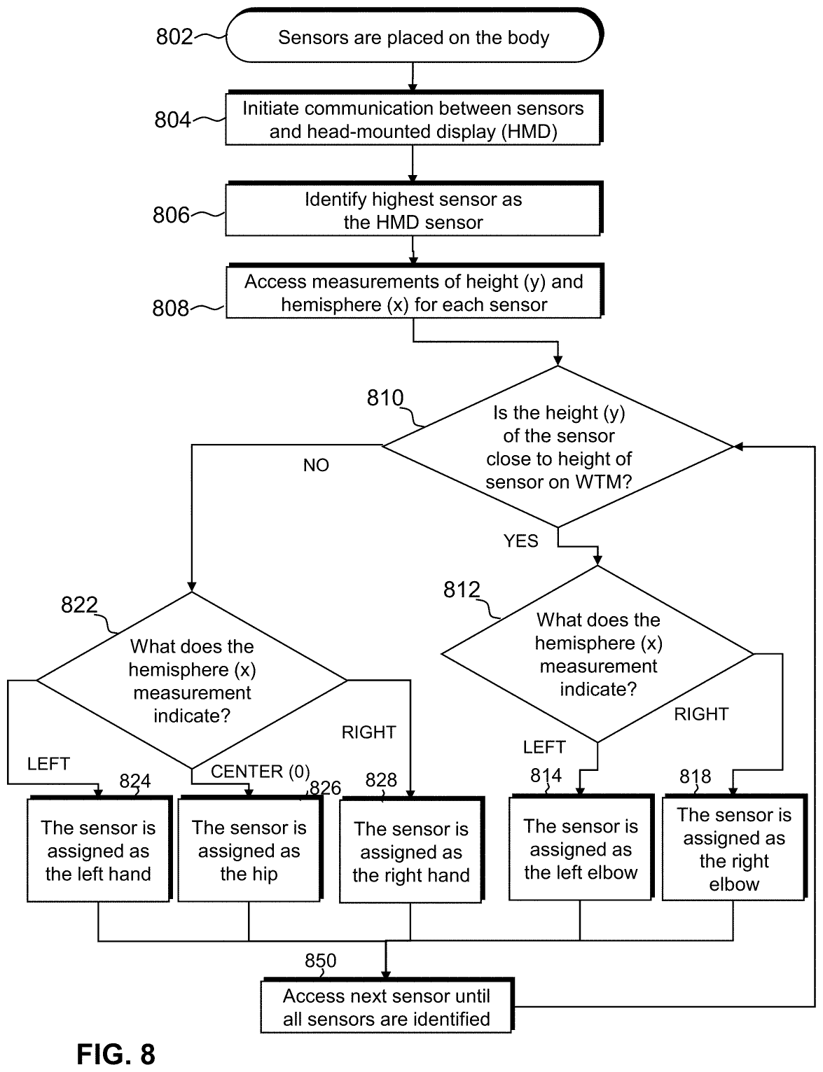

FIG. 8 depicts an illustrative flowchart of a process for automatic binding of sensors to the patient's body parts, in accordance with some embodiments of the disclosure. Some embodiments may utilize an avatar engine, e.g., as part of a VR application, stored and executed by one or more of the processors and memory of a headset, server, tablet, and/or other device.

The process begins after the sensors are placed on the body at step 802. In some embodiments, sensors may be applied to, for instance, the patient's head, back, hips, elbows, and wrists. In the process of FIG. 8, there are no leg sensors applied and the patient is sitting with her hands on her knees. In this process, a wireless transmitter module (WTM) includes a sensor and is worn on a sensor band that is laid over the patient's shoulders. In this process, the head-mounted display (HMD) includes a sensor, as well.

At step 804, the avatar engine initiates communication between sensors and the HMD. In some embodiments, each WSM wirelessly communicates its position and orientation with the HMD receiver. In some embodiments, the WTM sensor wirelessly communicates with the HMD receiver. In the examples in FIGS. 1B-C, 5, and 6, the sensor attached to the WTM on the patient's back is identified as sensor 202B and depicted with the other sensors 202.

At step 806, the avatar engine identifies the highest sensor as the sensor on the head-mounted display. In the example of the patient in FIG. 6, the sensor with the highest position is identified as the sensor 202A on the HMD 201.

At step 808, the avatar engine accesses measurements of height (y) and hemisphere (x) for each sensor. In some embodiments, hemisphere may be measured as, e.g., right or left side. In some embodiments, hemisphere may be a measurement of horizontal distance.

At step 810, the avatar engine determines if the height (y) of the sensor close to height of sensor on WTM. If the height (y) of the sensor is close to height of sensor on WTM, then the sensor is most likely one of two sensors at the elbow. Then, at step 812, the avatar engine determines which hemisphere (x) the measurement indicates. If the sensor's hemisphere (x) measurement indicates left then, in step 814, the sensor is assigned as the left elbow. If the sensor's hemisphere (x) measurement indicates right then, in step 818, the sensor is assigned as the right elbow. After assignment of the sensor, the next sensor is accessed, at step 850, until all sensors are identified.

If the avatar engine determines, at step 810, that the height (y) of the sensor is not close to height of sensor on WTM, then the avatar engine determines that it is one of the three sensors that are similar heights, e.g., the wrist sensors and the hip sensor. Then, at step 822, the avatar engine determines which hemisphere (x) the measurement indicates. If the sensor's hemisphere (x) measurement indicates left then, in step 824, the sensor is assigned as the left hand/wrist. If the sensor's hemisphere (x) measurement indicates center (e.g., close to 0) then, in step 826, the sensor is assigned as the hip. If the sensor's hemisphere (x) measurement indicates right then, in step 828, the sensor is assigned as the right hand/wrist. After assignment of the sensor, the next sensor is accessed, at step 850, until all sensors are identified.

At step 850, when the next sensor is accessed, until all sensors are identified, the process loops back to step 810 and the height is examined.

### Merging of Coordinate Systems in WTM Embodiments According to the Present Disclosure

Virtual Reality systems, such as one or more embodiments disclosed herein, may utilize multiple coordinate systems in order to, for example, identify and track body movements in a virtual world and a real world. Virtual reality systems endeavor to immerse the user deeply in a VR world with vision and motion tracking, so dependably simulating real world movements in a virtual world is essential. For instance, recreating the movements of a patient reaching for a virtual object, e.g., in a therapy session, may need feedback to see where her hand is in the virtual world as she moves her head to view the action. As disclosed herein, a VR system with several body sensors is able to better approximate a physical movement in virtual world, as well as provide data and feedback. Accordingly, such a system may need to convert between a physical world and a virtual world.

Some approaches in VR systems may only use one set of coordinates. Such an approach may animate avatars incorrectly. Such an approach may not be able to capture movement data for a therapy patient. Such an approach may not capture accurate measurements for therapy sessions. Here exists a need to use multiple coordinate systems in a VR world to, e.g., collect proper data accurately for use in a therapeutic setting.

As discussed in an earlier example, optical tracking and EM tracking may each have their own particular advantages and challenges. Optical tracking advantageously tracks real world movement in global coordinates but requires line of sight for tracking. Line of sight issues are especially problematic when tracking body movements, as body parts often overlap during ordinary movement in ways difficult for a camera to comprehend. EM tracking is advantageously not limited by line of sight, but it is self-referential. By merging these two coordinate systems, the system may track body position without requiring line of sight and may simultaneously track global coordinates. In this way, the best aspects of each individual coordinate system may be merged to generate a combination coordinate system with improved performance and usability.

In some embodiments, a special sensor called a wireless transmitter module (WTM), such as sensor 202 as depicted in FIG. 4B, is worn on the patient's back and represents the point of origin for the other sensors. One of these sensors, e.g., WSM 202A, is physically attached to the back of an HMD that utilizes six degrees of freedom and inside-out tracking. In such embodiments, self-referential and global coordinates may be merged by first querying the global coordinates for the location and position of the HMD in the VR world space. Since one of the sensors is physically attached to the back of the HMD, the position and rotation of that particular sensor can be determined based on its physical offset from the HMD. From the HMD's position relative to the WTM, the WTM position in the VR world space is calculated. The WTM position in the VR world space may be deduced from the inverse of the sensor position and the orientation. Some embodiments may calculate an inverse transform, for example, using four-by-four matrices and multiplying the matrix associated with the sensor mounted on the HMD with the matrix associate with the WTM. With the WTM's Position & Orientation calculated in virtual reality world space, we can now determine the location of the remaining sensors based on their relative P&O to the WTM. In this way, an EM tracking system and an optical tracking system may be merged into a combination coordinate system.

FIG. 9 depicts an illustrative flowchart of a process for merging WTM coordinates into the VR world space, in accordance with some embodiments of the disclosure. Some embodiments may utilize an avatar engine, e.g., as part of a VR application, stored and executed by one or more of the processors and memory of a headset, server, tablet, and/or other device.

The process begins after the sensors are placed on the body at step 552. As described in further detail below, the sensors are assigned to corresponding body parts. In some embodiments, sensors may be applied to, for instance, the patient's head, back, hips, elbows, and wrists. For example, wireless sensor modules (WSMs) are worn just above each elbow, strapped to each hand, and on a pelvis band that positions a sensor adjacent to the patient's sacrum on their back. In this process, a wireless transmitter module (WTM) includes a sensor and is worn on a sensor band that is laid over the patient's shoulders.

At step 554, the avatar engine initiates communication between sensors and the HMD. In some embodiments, each WSM wirelessly communicates its position and orientation with the HMD receiver. In some embodiments, the WTM sensor wirelessly communicates with the HMD receiver. In the examples in FIGS. 1B-C, 5, and 6, the sensor attached to the WTM on the patient's back is identified as sensor 202B and depicted with the other sensors 202.

At step 556, the avatar engine accesses the global coordinates for the location and position of the HMD in the VR world. In some embodiments, HMD tracking defines VR world space.

At step 558, the avatar engine accesses the known physical offset of the sensor on the back of the HMD. In this process, the HMD includes a sensor, as well. In the example of the patient in FIG. 5, the sensor on the HMD is identified as the sensor 202A on the HMD 201.

At step 560, the avatar engine determines virtual world position and orientation of sensor on the HMD based on its physical offset from the HMD. For instance, sensor 202A has a fixed position relative to the system's cameras 204 in FIG. 4A.

At step 562, the avatar engine determines relative position of the sensor on the HMD in relation to the sensor on the WTM. For instance, the WTM may transmit position and orientation data to the HMD receiver and a relative position to the HMD may be determined.

At step 564, the avatar engine determines VR world position of the sensor on the WTM based on position and orientation of the sensor on HMD. For instance, the sensor on the HMD can identify where it is relative to the WTM, so by taking the inverse of that position and orientation, the avatar engine can deduce the WTM virtual world space position. Some embodiments may calculate an inverse transform, for example, using four-by-four matrices and multiplying the matrix associated with the sensor mounted on the HMD with the matrix associate with the WTM.

At step 566, the avatar engine determines VR world position of other sensors based on their relative position to the sensor on the WTM. Some embodiments may use a composition of transformations to, e.g., take an offset and apply a second offset. For instance, the WTM may know and transmit its P&O data based on the offset to the HMD mounted sensor (e.g., the inverse of the offset), then all of the WSMs transmit their positions based on their offset to the WTM. With the WTM's Position & Orientation calculated in virtual reality world space, we can now determine the location of the remaining sensors based on their P&O relative to the WTM.

### Determining Body Movements Based on Sensor Data According to the Present Disclosure

Animating a virtual reality avatar based on sensor data may provide immediate feedback for a patient performing an action, but may provide valuable data for patient movement to, e.g., a therapist or medical professional. As disclosed herein, feedback for virtual reality activities may be provided in visual and data forms and many known body movements may have associated ranges of motion to be recorded and monitored.

In a virtual reality system where sensors are placed on a patient's body, e.g., at positions identified in FIG. 1C, the system does not necessarily capture data about a patient's range of motion directly. For instance, a single sensor may not be equipped to directly measure an angle of shoulder movement or the rotation of a forearm. Translation of sensor data into body movement data does not only animate a VR avatar, it can be used to reliably produce key patient movement data.

In some embodiments, such as physical therapy, a VR avatar may simulate real-life movement of a patient performing activities or exercises, but in a VR world. In some instances, it may be beneficial to perform such activities with the safety and supervision of a physical therapist. In many cases, the goals for physical therapy may be to restore or improve physical abilities of the patient to facilitate tasks in their daily life. Activities may be used to improve, e.g., range of motion, balance, coordination, joint mobility, flexibility, posture, endurance, and strength. For instance, with stroke victims, physical therapy with VR may involve tasks and exercises that imitate real-world activities such as reaching, grabbing, lifting, rotating, and other actions. Some key measurements of patient progress may be their range of motion for particular body parts and joints.

Generally, measurements that may be valuable for tracking may include flexion, extension, abduction, adduction, pronation, supination, and others. Flexion and extension are generally referred to as bending movements with flexion describing anterior (forward) movements and extension describing posterior directed movements. Extension is often viewed as straightening from a flexed position or bending backwards. Abduction refers to movement of a limb laterally away from the midline of a body or toward another body part. Adduction is generally thought of as the movement of a limb or other part toward the midline of the body or toward another part.

Using sensor position and orientation data, a VR system may be able to determine range of motion measurement such as cervical rotation, cervical flexion, cervical extension, shoulder flexion, shoulder extension, shoulder internal/external rotation, shoulder abduction, elbow flexion, elbow extension, forearm pronation, forearm supination, wrist flexion, and wrist extension, among others.

FIG. 10A is an exemplary diagram depicting participant wrist movement and potential range of motion within an illustrative system, in accordance with some embodiments of the disclosure. For instance, FIG. 10A depicts examples of patient wrist movement and illustrates potential measurements of range of motion. Range of motion measurements of wrist movement may be made from an axis of a wrist at rest.

Wrist flexion and extension are the bending movements of the wrist as illustrated in FIG. 10A. Wrist flexion describes anterior movement of the hand bending down to the wrist. Extension describes posterior-directed movement of the hand moving back toward the forearm.

Supination and pronation are movements of a forearm that go between two rotated positions. Pronation is the motion that moves the forearm from the supinated (anatomical) position to the pronated (palm backward) position. This motion is produced by rotation of the radius bone at the proximal radioulnar joint, while moving the radius at the distal radioulnar joint. The proximal radioulnar joint is a pivot joint that allows for rotation of the head of the radius. Supination is the opposite movement, where rotation of the radius returns the bones to a parallel position and moves the palm to the anterior facing (supinated) position.

Ulnar deviation, otherwise known as ulnar flexion, is the movement of bending the wrist to the little finger, or ulnar bone, side. Radial deviation, otherwise known as radial flexion, is the movement of bending the wrist to the thumb, or radial bone, side.

FIG. 10B is an exemplary diagram depicting participant wrist movement and potential range of motion within an illustrative system, in accordance with some embodiments of the disclosure. For example, FIG. 10B depicts an example of range of motion for patient wrist movement. In some embodiments, a sensor's position data may be measured and compared to a plane to determine an angle of movement from that plane. For instance, sensor 202 may be placed on the back of the patient's hand and can measure position and rotation data to the VR system in order to calculate an angle of the range of motion for the wrist.

FIG. 10B illustrates measurement of a range of motion for patient's wrist flexion to be 60 degrees. For example, when a patient's wrist bends forward, the range of motion measured from the plan of the wrist at rest is 60 degrees. FIG. 10B also illustrates measurement of a range of motion for patient's wrist extension to be 60 degrees. For example, when a patient's wrist bends backwards, the range of motion measured from the plan of the wrist at rest is 60 degrees. A wrist at rest is thought to be at 0 degrees extension or flexion.

FIG. 10C is an exemplary diagram depicting participant elbow movement and potential range of motion within an illustrative system, in accordance with some embodiments of the disclosure. For instance, FIG. 10C depicts an example of range of motion for patient elbow movement. Elbow flexion is considered bending of the arm inward at the elbow. Elbow extension is considered opening of a bent arm to a straight arm. In some embodiments, sensors' position data may be measured and compared to a plane to determine an angle of movement from that plane. In the case of the scenario presented in FIG. 10C, at least sensors at the hand and above the elbow are used to determine an angle of range of motion for the arm/elbow. Full elbow extension is typically thought of as 0 degrees, but some people may be able to hyper-extend beyond zero degrees.

FIG. 10D is an exemplary diagram depicting participant shoulder movement and potential range of motion within an illustrative system, in accordance with some embodiments of the disclosure. For example, FIG. 10D depicts an example of range of motion for patient shoulder movement. Shoulder flexion is considered forward lifting of the arm upward around the shoulder. Shoulder extension is considered rotating the arm downward or backwards. In some embodiments, sensors position data may be measured and compared to a plane to determine an angle of movement from that plane.

FIG. 10E is an exemplary diagram depicting participant shoulder movement and potential range of motion within an illustrative system, in accordance with some embodiments of the disclosure. For example, FIG. 10E depicts an example of range of motion for patient shoulder movement. Shoulder abduction is considered lifting of the arm upward around the shoulder to the side. Shoulder add is considered rotating the arm downward or backwards. In some embodiments, sensors position data may be measured and compared to a plane to determine an angle of movement from that plane.

### Automatically Correcting Sensor Orientation According to the Present Disclosure

Occasionally, when VR avatars are animated, a participant or observer may see movement of a virtual body part that does correlate to actual physical movement, indicating misplacement of a sensor. Such a mistake may not be caught until an avatar is rendered and animated.

As disclosed herein, a virtual reality system may automatically adjust and correct sensor orientation based on the sensor's relative position and orientation. For instance, a VR system may determine that an avatar could be hyperextending its elbow in a grotesque manner, based on a sensor placed backwards, and automatically correct the orientation of the sensor prior to rendering such an elbow bend. Such an effect of a patient seeing a representative avatar perform an impossible bending of a joint is not likely conducive to therapy. Waiting until rendering and animation of an avatar before identifying the mistake costs therapy time. Accordingly, a virtual reality system monitoring and correcting sensor orientation is needed.

In some embodiments, the system detects when a sensor has been mistakenly strapped onto a wearer backwards and/or upside down. This may be particularly important in embodiments where the system provides patient therapy to allow patients to use the system as quickly as possible. Once this is detected, the system automatically compensates for the incorrect placement so the session can continue without having to remove and reattach the sensor to the patient. Automatically correcting may including inverting one of the axes of the problematic sensor. In some embodiments, orientation of the sensor is corrected before animating an avatar, e.g., in a physically impossible pose. This feature may beneficially allow the sensors to not only be placed on any position of the body (as discussed earlier) but also allow the sensors to be attached in any orientation, thereby further increasing ease of use and reducing setup time.

Once sensors are bound to respective body parts, the system starts moving the virtual world avatar's skeletal nodes. With the avatar beginning to reflect movement of the patient's hands, elbow, pelvis, and head, the system evaluates if a skeletal node seems rotated outside the range of what is physically possible in the real world. If any measurement falls outside of this range, an orientation offset is applied to virtually flip the sensor in the opposite direction on the yaw axis. The hands are the first to be evaluated. The system measures the angle of the avatar skeleton's left- and right-hand nodes. If the resulting angle is greater than 55 degrees, the system flags this sensor as having been put on backwards and its rotation value is now flipped along the yaw in the opposite direction. The system next evaluates the pelvis sensor by measuring the angle of the avatar skeleton's spine node. If the resulting angle is greater than 50 degrees, the system assumes that this sensor was placed upside down and flips the sensor's yaw rotation in the opposite direction.

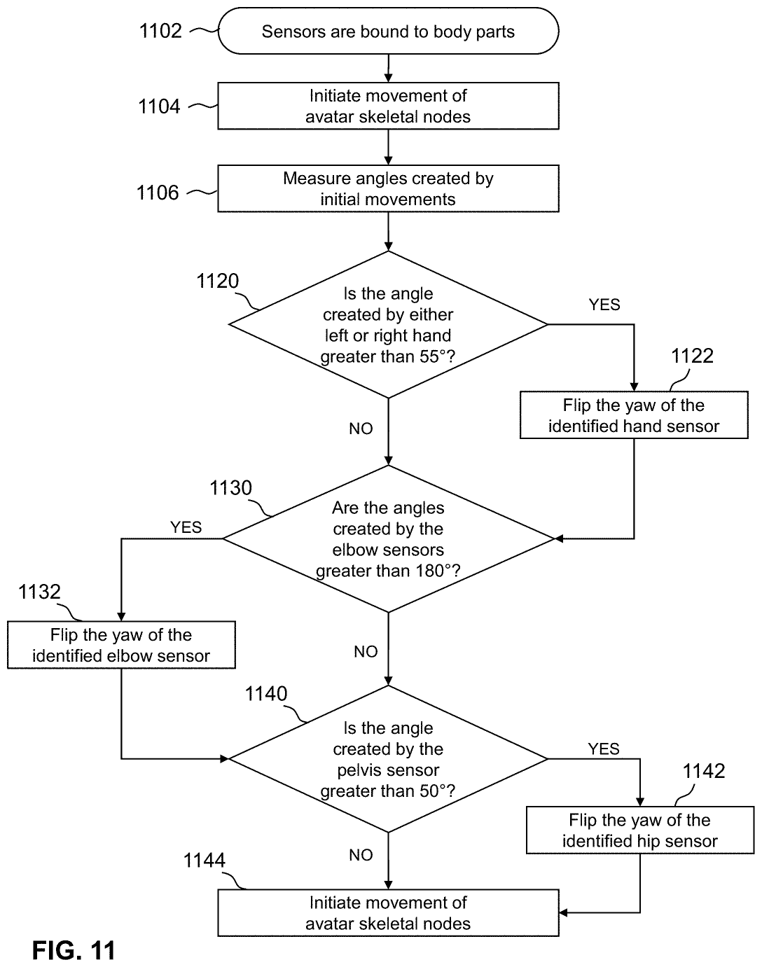

FIG. 11 depicts an illustrative flowchart of a process for automatically correcting sensor orientation, in accordance with some embodiments of the disclosure. Some embodiments may utilize an avatar engine, e.g., as part of a VR application, stored and executed by one or more of the processors and memory of a headset, server, tablet, and/or other device.

The process begins after the sensors are placed on the body at step 1102. In some embodiments, sensors may be applied to, for instance, the patient's head, back, hips, elbows, and wrists. In the process of FIG. 11, the patient is sitting at rest with her hands on her knees. In this process, a wireless transmitter module (WTM) includes a sensor and is worn on a sensor band that is laid over the patient's shoulders. In this process, the head-mounted display (HMD) includes a sensor, as well.

At step 1104, the avatar engine initiates movement of the avatar skeletal nodes. In some embodiments, each sensor communicates its position and orientation with the WTM. The avatar skeletal nodes are moved based received communications from the sensors to the WTM.

At step 1106, the avatar engine measures the angles created by initial movements of the avatar. In some embodiments, the movements are not yet animated so that the avatar does not depict poses that are physically impossible.

At step 1120, the avatar engine determines if the angle created by either left or right hand greater than, e.g., 55°. In some embodiments, a 55-degree angle is used as the threshold, but the threshold may be different or more precise. If the avatar engine determines the angle created by either hand is greater than the threshold then the yaw axis of the hand sensor is inverted, at step 1122. After step 1122, or if the avatar engine determines the angle created by either hand is less than or equal to the threshold, the process advances to step 1130.

At step 1130, the avatar engine determines if the angle created by either left or right elbow is greater than, e.g., 180°. In some embodiments, a 180-degree angle is used as the threshold, but the threshold may be different or more precise. If the avatar engine determines the angle created by either elbow is greater than the threshold then the yaw axis of the elbow sensor is inverted at step 1132. After step 1132, or if the avatar engine determines the angle created by either elbow is less than or equal to the threshold, the process advances to step 1140.

At step 1140, the avatar engine determines if the angle created by the hip is greater than, e.g., 50°. In some embodiments, a 50-degree angle is used as the threshold, but the threshold may be different or more precise. If the avatar engine determines the angle created by the hip is greater than the threshold then the yaw axis of the elbow sensor is inverted at step 1132. After step 1142, or if the avatar engine determines the angle created by the hip is less than or equal to the threshold, the process advances to step 1144, and movement of the avatar skeletal nodes proceeds.

### Generating Avatar Structure According to the Present Disclosure

One of the keys to providing feedback and data through virtual reality is realistic animation of a VR avatar based on sensor data from a patient's real-world body movements. In a virtual reality system where sensors are placed on a patient's body, e.g., at positions identified in FIG. 1C, the system may use sensor data to, e.g., interpolate body positions and animate an avatar.

The avatar may be comprised of virtual bones, a virtual skin or mesh, and/or virtual clothes. Immersion requires a faithful mapping of the user's movements to the avatar. The user controls the avatar by moving their own body, and thus the avatar may be able to mimic every possible motion the user performs. Having a pre-recorded motion for every possible position is either impractical or impossible. Instead, the animations may be rendered from a set of tools, whose use allows on demand rendering. In some embodiments, systems and methods of the present disclosure utilize numerous pre-recorded 3D models called key poses. The key poses are typically polygon renders of an avatar defined by a plurality of vertices.

A user's position at a given point in time is rendered by blending the nearest key poses in proportion to their proximity to the user's tracked position, e.g., vertex animation. In some embodiments, systems and methods of the present disclosure utilizes a skeleton rig, whereby the bones of the skeleton rig are manipulated in the rendering process to position the avatar in a position similar to the user's own position. In alternate embodiments, a combination of vertex and skeletal animation is applied to render an avatar.

In one example, the avatar includes virtual bones and comprises an internal anatomical structure that facilitates the formation of limbs and other body parts. Skeletal hierarchies of these virtual bones may form a directed acyclic graph (DAG) structure. Bones may have multiple children, but only a single parent, forming a tree structure. Two bones may move relative to one another by sharing a common parent.

Virtual skin may surround the virtual bones as an exterior surface representation of the avatar. The virtual skin may be modeled as a set of vertices. The vertices may include one or more of point clouds, triangle meshes, polygonal meshes, subdivision surfaces, and low-resolution cages. In some embodiments, the avatar's surface is represented by a polygon mesh defined by sets of vertices, whereby each polygon is constructed by connecting at least three vertices.

Each individual vertex of a polygon mesh may contain position information, orientation information, weight information, and other information. The vertices may be defined as vectors within a Cartesian coordinate system, whereby each vertex has a corresponding (x, y, z) position in cartesian space. In alternative embodiments, the virtual bone transformations may be defined as vectors in quaternion space, whereby each bone has a corresponding (1, i, k, j) position in quaternion space. Quaternion representation of rotation for bone transformations beneficially avoids gimbal locks that temporarily reduces a tracked object's degrees of freedom. Gimbal lock is associated with tracking, and, thus, animation errors.

The movement of the avatar mesh vertices with the skeletal structure may be controlled by a linear blend skinning algorithm. The amount each vertex is associated with a specific bone is controlled by a normalized weight value and can be distributed among multiple bones. This is described more fully in the Skeletal Animation section below.

The surface of the avatar is animated with movement according to either vertex animation, skeletal deformation, or a combination of both. Animation techniques include utilization of blendspaces which can concurrently combine multiple drivers to seamlessly and continuously resolve avatar movement. An example of using a blendspace is a strafing movement model that controls foot animation based on avatar forward/backward and left/right movement. Another example is four hand shapes representing finger positions with different wrist or metacarpus positions (in, out, up, down). In both examples each shape or animation pose is blended together depending on the degree to which its driver is currently active, i.e. how much the avatar has moved in world space or the currently tracked position of the wrist. Morph target shapes are stored offsets of affected vertices that can be blended in and combined with skeletal deformation to create more convincing deformation. An example of morph target animation is the bulging of a bicep muscle in response to forearm movement. Key pose interpolation is the skeletal movement of the avatar blending sequentially from pose to pose where the poses are defined by an animator setting key frame values on the bone transforms.

### Animating an Avatar Skeleton According to the Present Disclosure

As disclosed herein, a virtual reality system may animate a patient's representative avatar in order to, e.g., give feedback of the patient's motion and facilitate action in the VR application activities.

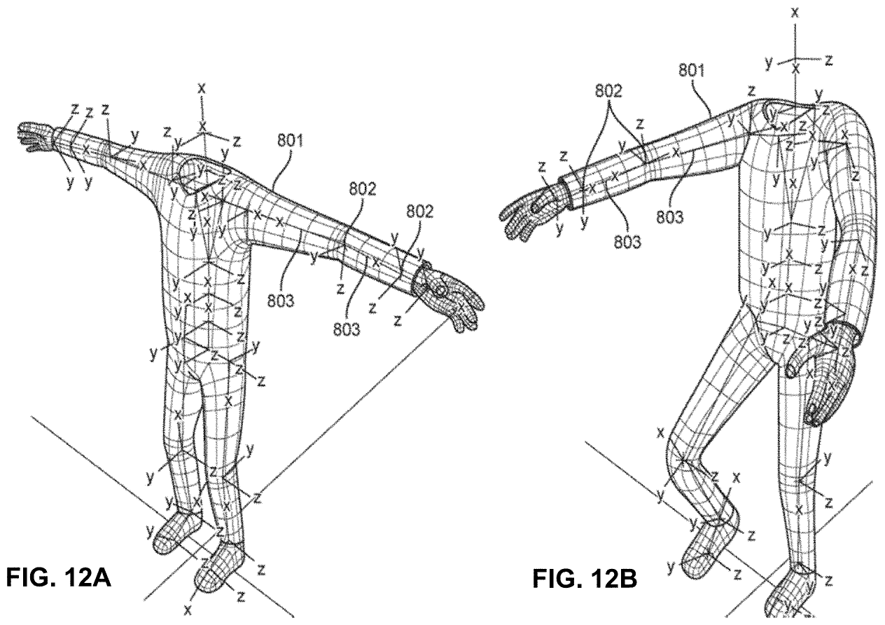

FIGS. 12A and 12B each illustrate a 3D model comprised of a mesh fitted with a skeleton, in accordance with some embodiments of the disclosure. These figures show the mesh 801 as a framework and the skeleton as a hierarchy of pivot points 802 labeled with X, Y, and Z axes where the lines 803 between them indicate the parenting relationship of the bones. Alternatively, these pivot points 802 are labeled with (1, i, k, j) axis labels, which correspond to quaternion coordinates. Each axis may be characterized as a mathematical vector. The parenting relationship allows bones to inherit the motion of their parent bones. The bones of the virtual skeleton may or may not precisely mimic the joints seen in typical human anatomy.

Each bone of the skeleton forms a transformation which influences all vertices associated with the bone. The amount of influence each bone has on each vertex is controlled by a weighting system. For a vertex animation approach, the skeleton of a 3D model is manually manipulated across the joints (or pivot points) to form particular poses of the 3D model. These poses are sometimes called deformations, in that they are deformations of the original 3D model. These deformations are saved as offsets or deltas from the original model in order to be used as key poses for a vertex animation approach.

### Procedural Hand Animation According to the Present Disclosure

To appear realistic, finger poses may be proportionally blended between one or more poses. These poses are achieved as keyframes on the bones which deform the fingers. For hand animations, this means that finger movement animations may be animated both in proportion to wrist or metacarpus movement and with the same directionality. This movement is achieved by applying a driver mechanism across five poses in a two-dimensional BlendSpace which includes a neutral pose when no driver mechanism is active. The driver mechanism may execute a mathematical transformation that generates a hand pose that is linearly related to the degree of wrist flexion or has a curved relation to the degree of wrist flexion. In the case of linear relationship between wrist flexion and finger movement, 25% of wrist flexion from neutral may cause an animation that is 25% deformed towards said key pose and 75% deformed towards the neutral pose. If wrist flexion is angled towards more than one key pose, then hand animations are interpolated proportionate to the proximity of nearby key poses and the neutral pose. For instance, a wrist flexion measurement of 33% “in” and 33% “up” may cause the generation of a hand animation that is interpolated evenly between the hand model's neutral pose, “in” pose, and “up” pose. This middle pose exists within the blend space of these three individual poses.

A curved relationship between wrist flexion and finger movement may generate a different animation for a given wrist flexion when compared to a model utilizing a linear relationship. Assume a hand is moving from the neutral pose to an “in” pose. During the first 25% of wrist flexion, the animation may traverse half the blend space and produce an animation that is 50% “in” and 50% neutral. In this way, the animation driver is accelerated at the front end; showing half the of the hand model's blend space for the first quarter of wrist flexion. The remaining half of the blend space is then slowed down on the backend and spread out across three quarters of wrist flexion. Of course, this approach may be reversed and hand animations may be slowed on the frontend and accelerated on the backend.

Such an approach may also utilize easing functions to accommodate rapid movements. Rapid movements may cause an animation technique to temporarily lose accuracy by improperly animating extreme hand poses. Thus, the rate at which a hand may enter or leave a pose is limited by an ease function. The ease functions act to temporarily slow down the display of animated movements. In essence, the ease function generates a lag time in reaching a particular pose when movements are deemed too rapid. In addition, the ease function may avoid animation jerks from gimbaling events that can occur during Cartesian coordinate rotations.

### Customization of an Avatar Solver According to the Present Disclosure

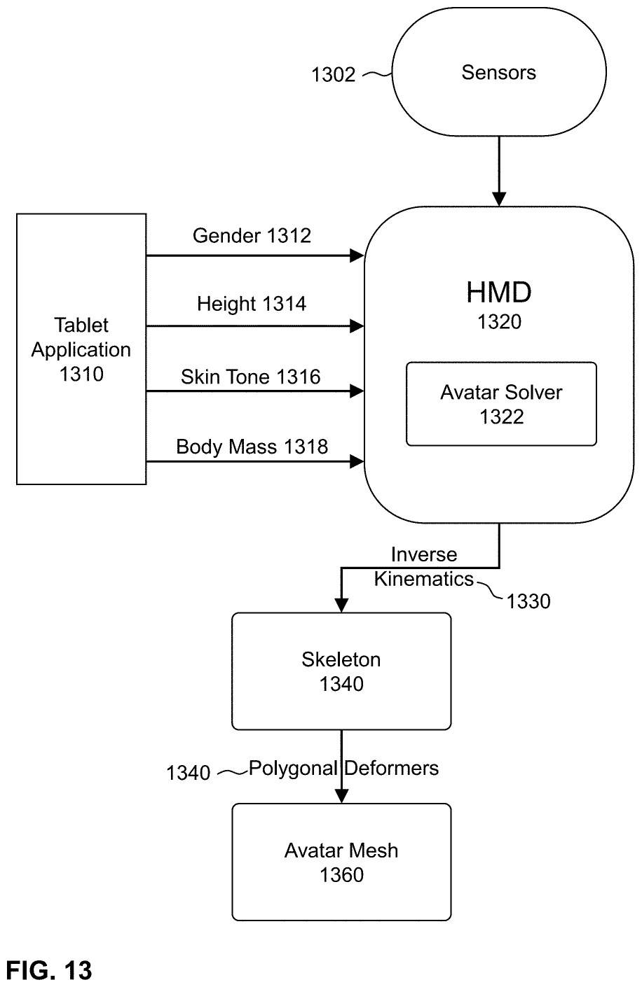

FIG. 13 depicts an illustrative flow diagram of a process for creating an avatar mesh, in accordance with some embodiments of the disclosure. In some embodiments, the HMD software 1320 includes an avatar solver 1322 employing inverse kinematics (IK) 1330 and a series of local offsets to constrain skeleton 1340 of the avatar to the position and orientation of the sensors. Skeleton 1340 then deforms a polygonal mesh 1340 to approximate the movement of the sensors.

As depicted in FIG. 13, for example, tablet application 1310 may edit and store, for example, four avatar values that alter the appearance of the avatar, such as, gender 1312, height 1314, skin tone 1316, and body mass 1318. These values get communicated from tablet application 1310 to HMD 1320 (in some embodiments, on startup), so the avatar displayed on the HMD matches the patient. Gender 1312 value determines the base polygonal mesh to be displayed (female or male). In some embodiments, gender 1312 value also determines a Blend Space used to, for example, support the rendering of the avatar's hand posing for various types of HMDs (e.g., Oculus™ or Sixense™) based on wrist orientation. Height 1314 value scales the polygonal model and its deforming skeleton 1340 to the appropriate size. Skin Tone 1316 value determines the color of the visible skin on the hands and arms of the model. Body mass 1318 alters the shape of the avatar mesh 1360 to approximate the patient. Body mass 1318 may be, e.g., a measure from 0 to 1 of a patient's body mass, where 0 represents the smallest patient and 1 represents the largest patient based on, e.g., average body sizes and shapes. Body mass 1318 may be, for instance, based on a measure of body mass index (BMI). In some embodiments, data for body mass 1318 may alter the size or shape of skeleton 1340.

In some embodiments, there may be known offsets that define the distance from the positions of the physical sensors (WSMs) to the avatar skeleton IK elements. These include, for example, the offset from the hand-worn WSM to the end-effector of the arm two-bone IK solver, the offset from the pelvis sensor to the avatar hip bone, and the offset from the WTM to the top of the avatar spine. These exemplary offsets may be proportionately scaled to accommodate the variance of patient sizes as determined by, e.g., gender 1312, height 1314, and/or body mass 1318 data. For example, a tall male with high body mass may have each of those offsets proportionately increased to reflect the patient data. This allows fine tuning of how the sensor system drives the customized avatar skeleton to create avatar upper body movement and appearance that mimics, as closely as possible, patient movement and appearance, as well as maximizing the accuracy of range of motion (ROM) data.

In a vertex animation approach, movement animations may be executed as interpolations between morph targets. A morph target is a new shape created by a copy of the original polygonal mesh with vertex order and topology being maintained and then moving the vertices to create the new desired shape. The morph target is then saved as a set of 3D offsets, one for each vertex, from the original position to the new target position of that vertex. Every deformation made of the model to be animated exists as a key pose or morph target across a variety of triggering mechanisms. For the animation of a hand, movement is animated as an interpolation between the neutral shape and the one or more target shapes. At a basic level, applying a morph target is moving each vertex linearly towards its target shape in the direction of the saved offset vector. The amount of activation of the blendshape is controlled by its weight. A weight of 1.0 activates the full target shape. A weight of 0.5 would move each vertex exactly halfway towards the target position. Multiple blendshape targets can be active at once with each controlled by its own weight value. As the weight of blendshapes change over time, smooth interpolation between intermediate shapes is achieved.

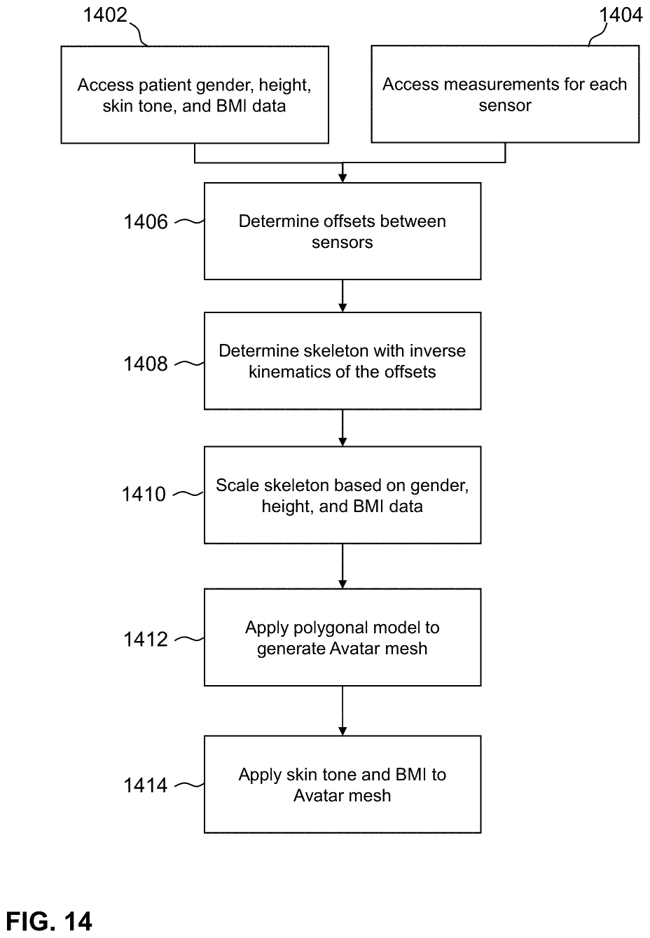

FIG. 14 depicts an illustrative flowchart of a process for creating and scaling an avatar mesh, in accordance with some embodiments of the disclosure. Some embodiments may utilize an avatar engine, e.g., as part of a VR application, stored and executed by one or more of the processors and memory of a headset, server, tablet, and/or other device.

At step 1402, the avatar engine accesses patient gender, height, skin tone, and body mass data. In some embodiments, a tablet application can edit and store four avatar values that alter the appearance of the avatar, e.g., gender, height, skin tone, and body mass. In some embodiments, a tablet application, e.g., in communication with the cloud server, may enable edit and store more than four (or fewer than four) avatar values that may alter the appearance of the avatar.

At step 1404, the avatar engine accesses measurements for each sensor. In some embodiments, such as depicted in FIG. 6, sensors 202 placed on the body provide P&O data relative to a sensor 202B with an emitter worn on a user's back (e.g., in a wireless transmitter module (WTM)).

At step 1406, the avatar engine determines offsets between sensors. For instance, these offsets may be distances from the positions of the physical sensors (WSMs). In some embodiments, distances between each hand and corresponding elbow may be measured. In some embodiments, the distance between the hip and the WMT may be measured. In some embodiments, the distance between the HMD and the WMT may be measured.

At step 1408, the avatar engine determines an avatar skeleton with inverse kinematics of the offsets. In some embodiments, the avatar skeleton may be created based on measurements between sensors.

At step 1410, the avatar engine scales the skeleton based on gender, height, and body mass data. For instance, some embodiments allow fine tuning of the customized avatar skeleton to create avatar upper body movement and appearance that closely mimics patient movement and appearance.

At step 1412, the avatar engine applies polygonal model to generate an avatar mesh.

At step 1414, the avatar engine applies skin tone and body mass data to an avatar mesh. For instance, the avatar engine may alter the build of the chest and arms based on the body mass data. In some embodiments, scaling the skeleton and fine tuning the appearance may maximizing the accuracy of range of motion (ROM) data.

### Determining, Storing, and Displaying Range of Motion Data According to the Present Disclosure

As discussed above, some embodiments may calculate and save participant (e.g., patient) range of motion (ROM) data in a database in the cloud. In some embodiments, this may be accomplished by evaluating the various skeletal nodes of the virtual avatar (using an avatar renderer, such as the Unreal Engine) and then calculating the resulting angle in a two-dimensional space.

In one example, a function in the HMD measures the bone angle on a specific axis using three inputs: the current rotation of the skeletal node relative to its parent node, an offset vector (e.g., forward, up, left, etc.), and a single plane. The function defines three different transforms to measure against: transform A, transform B, and transform C. Transform A represents the orientation of the skeletal node provided to the function, usually relative to its parent node. Transform B represents a location offset from transform A, taking into account the rotation from Transform A and the offset vector provided to the function. Transform C represents a location on the plane given to the function. The function determines the angle by (a) calculating the delta between transform A and transform B, as well as the delta between transform B and transform C; (b) taking the dot product of the two normalized resulting vectors; (c) calculating the arccosine value; and (d) converting the value from radians to degrees.

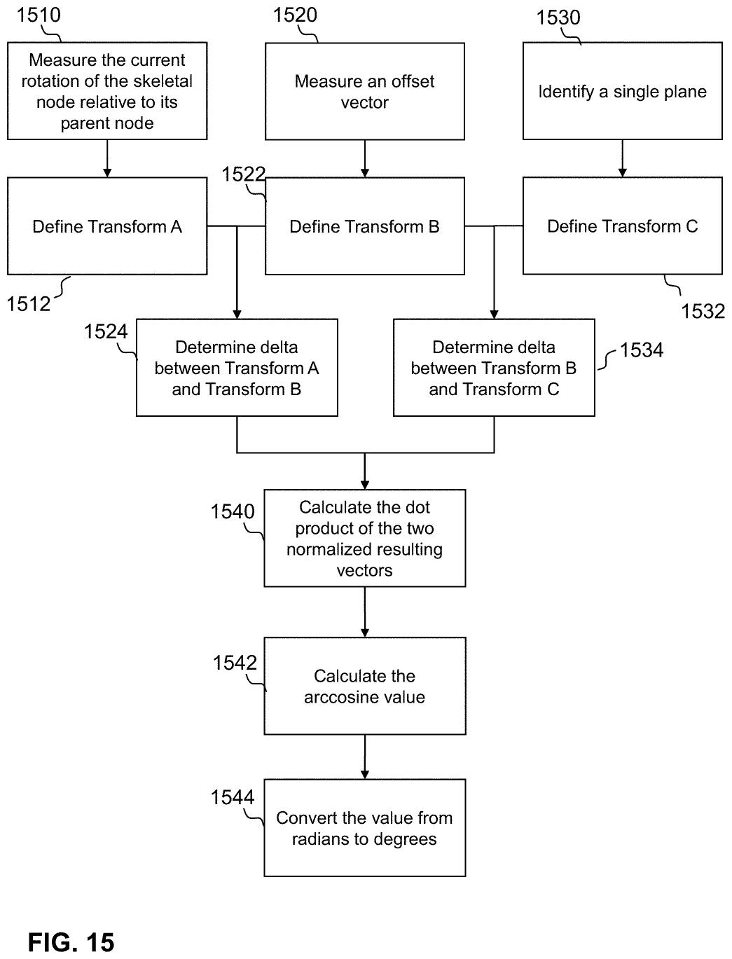

FIG. 15 depicts an illustrative flowchart of a process for measuring and storing range-of-motion (ROM) data, in accordance with some embodiments of the disclosure. Some embodiments may utilize an avatar engine, e.g., as part of a VR application, stored and executed by one or more of the processors and memory of a headset, server, tablet, and/or other device.

At step 1510, the avatar engine measures the current rotation of the skeletal node relative to its parent node. For example, the avatar engine measures the bone angle on a specific axis using the current rotation of the skeletal node relative to its parent node.

At step 1512, the avatar engine defines a Transform A vector based on the current rotation of the skeletal node relative to its parent node. In some embodiments, Transform A represents the orientation of the skeletal node provided to the function, usually relative to its parent node.

At step 1520, the avatar engine measures an offset vector. For example, the avatar engine measures the bone angle on a specific axis using an offset vector (e.g., forward, up, left, etc.).

At step 1522, the avatar engine defines a Transform B vector based on the current rotation of the offset vector. In some embodiments, Transform B represents a location offset from transform A, taking into account the rotation from Transform A and the offset vector provided to the function.

At step 1530, the avatar engine identifies a single plane. For example, the avatar engine measures the bone angle on a specific axis using a single plane.

At step 1532, the avatar engine defines a Transform C vector based on the single plane. In some embodiments, Transform C represents a location on the plane given to the function.

At step 1532, the avatar engine determines the delta between Transform A and Transform B.

At step 1534, the avatar engine determines the delta between Transform B and Transform C.

At step 1540, the avatar engine calculates the dot product of the two normalized resulting vectors. Geometrically, the dot product is the product of the Euclidean magnitudes of the two vectors and the cosine of the angle between them. The dot product results in a scalar.

At step 1542, the avatar engine calculates the arccosine of the result of the dot product. The arccosine, or inverse cosine, of the dot product produces an angle (typically, in radians).

At step 1542, the avatar engine converts the result from radians to degrees to provide a range of motion.

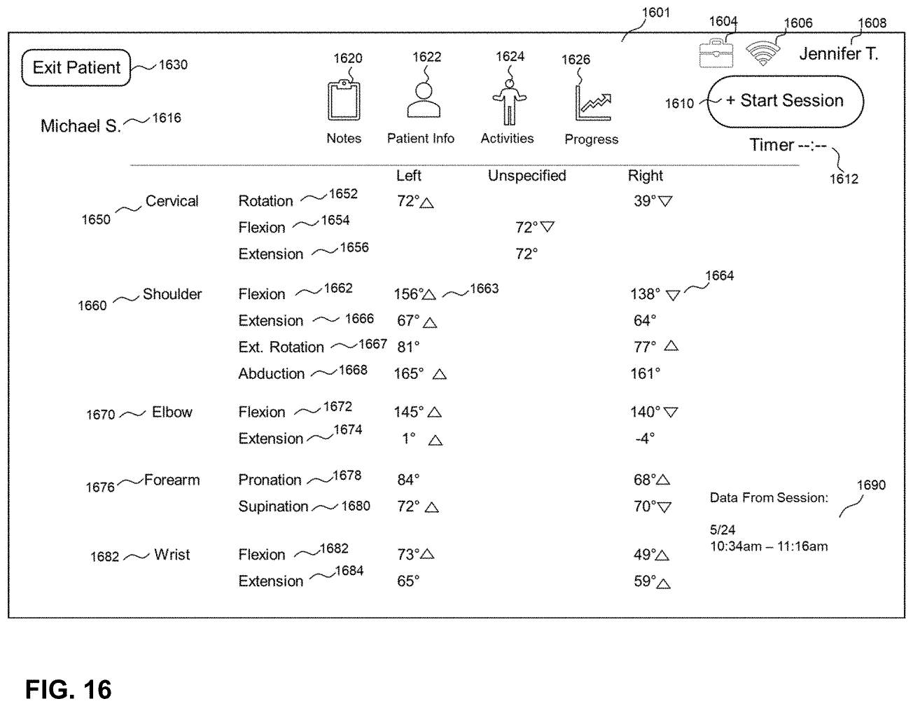

Using the avatar rendering engine, the system calculates the value for various range of motion for the wearer using the rotation and position data of each skeletal node, e.g., cervical rotation, cervical flexion, cervical extension, shoulder flexion, shoulder extension, shoulder internal/external rotation, shoulder abduction, elbow flexion, elbow extension, forearm pronation, forearm supination, wrist flexion, and wrist extension. These values are stored in, for example, a relational database by the GraphQL Server 364 depicted in FIG. 3. These values may be displayed on the therapist's tablet in a user interface such as depicted in FIG. 16.

FIG. 16 depicts an exemplary supervisor interface for an illustrative system, in accordance with some embodiments of the disclosure. For instance, FIG. 16 illustrates a user interface, user interface 1601, for use by an operator or therapist, after at least two therapy sessions using a VR system. User interface 1601 illustrates, for example, measurements for ranges of motion of various body parts and joints for a patient during a therapy session. With respect to FIG. 4, for instance, web browser 328 of tablet 320 may access file server 352 after logging in via Authorization and API Server 362 to access databases served via GraphQL server 364.

In the scenario depicted in user interface 1601, the therapy session was on May 24th, from 10:34 am −11:16 am, according to session timestamp 1690. Some embodiments may use arrows such as up arrow 1665 and/or down arrow 1666 to indicate changes in measurements since a prior session. In some embodiments, arrows to compare prior session data may be hidden. In some embodiments, such as instances with a new patient and/or if there were no prior data, arrows may not be presented.

In some embodiments, user interface 1601 depicts therapist identifier 1608 to designate which therapist signed into the system. User interface 1601 may depict wireless icon 1606 to denote a wireless network connection is established. Wireless icon 1606 may depict different strength levels based on the strength of the signal. Wireless icon 1606 may indicate connection to the VR system. Briefcase icon 1604 may take a user to settings. Patient name 1616 indicates the name of the patient whose data is displayed. User interface 1601 depicts exit patient button 1630, e.g., in the top left corner of the interface. Exit patient button 1630 may allow a therapist to change between patients. Each patient's data may be kept separately for privacy.

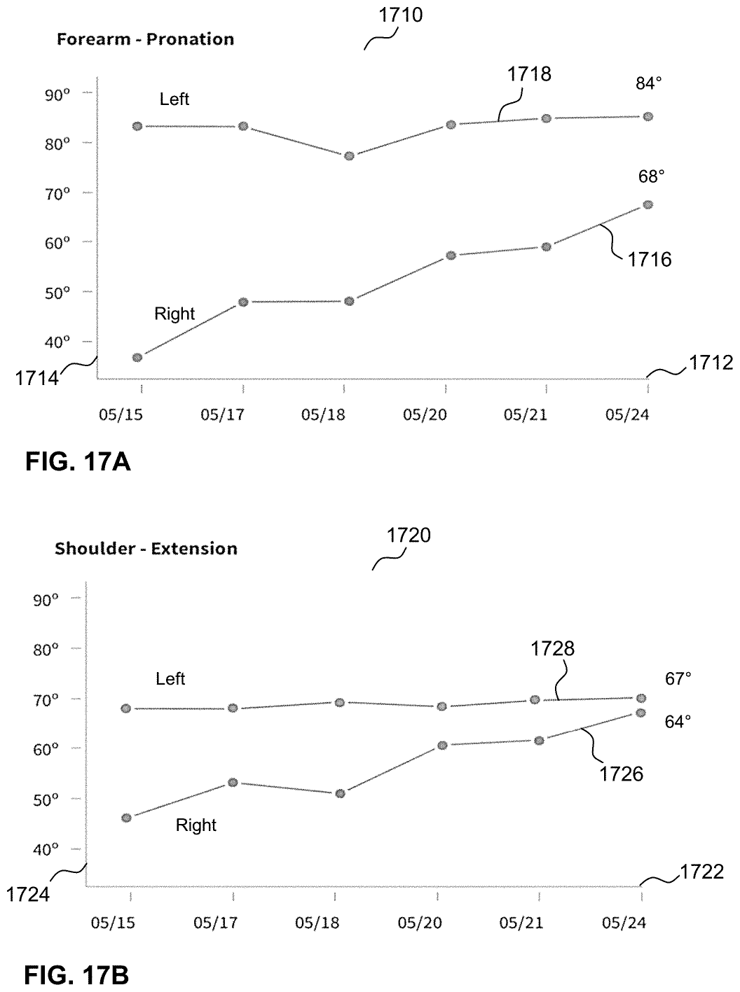

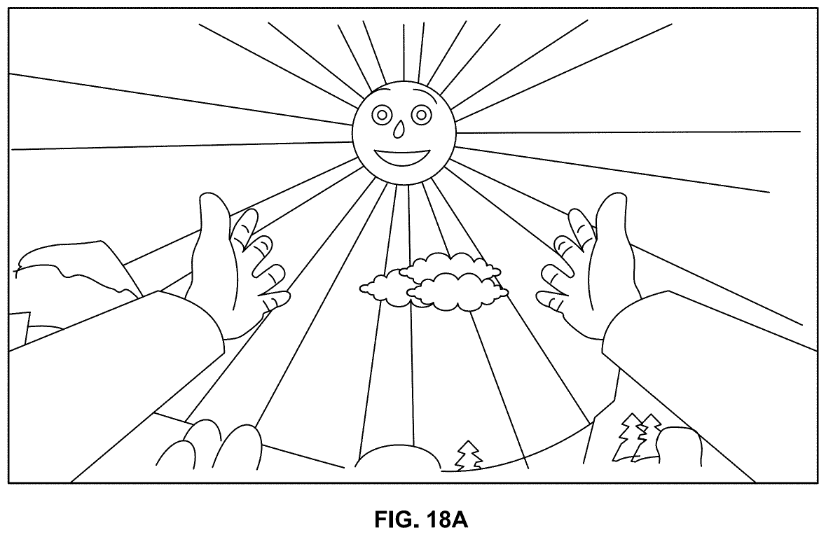

In some embodiments, such as depicted in user interface 1601, there may be a set of icons at the top of the interface. In some embodiments, each icon opens or switches to a particular tab. For instance, notes icon 1620 may switch to a notes tab for a summary of session and activity times. In some embodiments, notes icon 1620 may allow a therapist an interface to add notes for the particular patient. In some embodiments, patient info icon 1622 may allow a therapist to see an interface with additional patient info. Activities icon 1624 may reveal an interface to select and initiate activities for the patient wearing the VR headset and sensors. Progress icon 1626 may switch the interface to a chart view. In some embodiments, a chart view may include, e.g., line charts as depicted in FIGS. 17A and 17B.

User interface 1601 depicts start session button 1610. Start session button 1610 may begin a new session for the current patient, patient name 1616. Start session button 1610 may begin a session timer such as timer 1612. In some embodiments, starting a new session may clear the angles on the screen and indicate new measurements from the current session. In some embodiments, activities icon 1624 may allow changing of VR activities for the patient during the session.

Exemplary user interface 1601 may depict range of motion (ROM) data for several body parts and joints. ROM data may be depicted in a column for left body parts, right body parts, or unspecified/center body parts. ROM data may be generated based on sensor data during session activities and stored in a database, such as, e.g., Analytics 351, Relational Database 353, Logs 355, or other databases depicted in FIG. 4.

In some embodiments, user interface 1601 may provide range of motion information for cervical movement 1650, e.g., cervical rotation 1652, cervical flexion 1654, and cervical extension 1656. User interface 1601 may depict measurements, for instance, of cervical rotation 1652 as 72 degrees to the left and 39 degrees to the right. Cervical flexion 1654 and cervical extension 1656 are up and down movement and have no left or right values and are each measured as 72 degrees for this session.

User interface 1601 may also depict data for several motions for the shoulder 1660, such as shoulder flexion 1662, shoulder extension 1666, shoulder external rotation 1667, and shoulder abduction 1668. Shoulder flexion, e.g., as depicted in FIG. 10D, is considered forward lifting of the arm upward around the shoulder. Shoulder extension, e.g., as depicted in FIG. 10D, is considered rotating the arm downward or backwards. In this scenario, shoulder flexion 1662 is measured to be 156 degrees for the left shoulder and 138 degrees for the right shoulder for the session. Some embodiments may use arrows such as up arrow 1663 or down arrow 1664 to indicate progress (or lack of progress) in range of motion from therapy sessions. For instance, up arrow 1663 may indicate that shoulder flexion 1662 measured to be 156 degrees for the left shoulder is an improvement. Down arrow 1664 may indicate that shoulder flexion 1662 measured to be 138 degrees for the right shoulder is a decline in range of motion. In this scenario, shoulder extension 1666 is measured to be 67 degrees for the left shoulder and 64 degrees for the right shoulder for the session.

User interface 1601 depicts several motions for the elbow including elbow flexion 1672 and elbow extension 1674. Elbow flexion, e.g., as depicted in FIG. 10C, is considered bending of the arm inward at the elbow. In this scenario, elbow flexion 1672 is measured to be 145 degrees for the left elbow and 140 degrees for the right elbow for the session. Elbow extension, e.g., as depicted in FIG. 10C, is considered opening of a bent arm to a straight arm. In this scenario, elbow extension 1674 is measured to be 1 degree for the left elbow and −4 degrees for the right elbow for the session. Full elbow extension is typically thought of as 0 degrees, but some people may be able to hyper-extend beyond zero degrees.

User interface 1601 depicts several motions for the forearm including forearm pronation 1678 and forearm supination 1680. Forearm pronation, e.g., as depicted in FIG. 10A, is the motion that moves the forearm from the supinated (anatomical) position to the pronated (palm backward) position. In this scenario, forearm pronation 1678 is measured to be 84 degrees for the left arm and 68 degrees for the right arm for the session. Forearm supination, e.g., as depicted in FIG. 10A, is the opposite movement, where rotation of the radius returns the bones to a parallel position and moves the palm to the anterior facing (supinated) position. In this scenario, forearm supination 1680 is measured to be 72 degrees for the left arm and 70 degrees for the right arm for the session.

In some embodiments, user interface 1601 may depict several motions for the wrist including wrist flexion 1682 and wrist extension 1684. Wrist flexion, e.g., as depicted in FIGS. 10A and 10B, describes anterior movement of the hand bending down to the wrist. In this scenario, wrist flexion 1682 is measured to be 73 degrees for the left wrist and 49 degrees for the right wrist for the session. Wrist extension, e.g., as depicted in FIGS. 10A and 10B, is considered posterior-directed movement of the hand moving back toward the forearm. In this scenario, wrist extension 1684 is measured to be 65 degrees for the left wrist and 59 degrees for the right wrist for the session. A wrist at rest is thought to be at 0 degrees extension or flexion.

FIGS. 17A and 17B illustrate range of motion charts from a user interface for use by an operator or therapist after a therapy session using a VR system in accordance with one or more embodiments.

FIG. 17A depicts a chart from an exemplary supervisor interface for an illustrative system, in accordance with some embodiments of the disclosure. For instance, FIG. 17A illustrates a user interface featuring a range of motion chart for forearm pronation. Pronation is the motion that moves the forearm from the supinated (anatomical) position to the pronated (palm backward) position, as depicted in FIG. 10A. The interface depicted in FIG. 17A may be from a user interface for use by an operator or therapist after several therapy sessions using a VR system in accordance with one or more embodiments.

FIG. 17A includes line chart 1710 featuring a vertical and a horizontal axis. In line chart 1710, the horizontal axis is date axis 1712 which includes dates ranging from May 15 to May 24. In line chart 1710, the vertical axis is angle axis 1714 which includes degrees representing range of motion from about 40 degrees to 90 degrees. In some embodiments, the axes may be scaled differently. In some embodiments, range of motion measurements for each side of the body (e.g., left or right) may be depicted separately.

Line chart 1710 features two lines, line 1716 and line 1718, which correspond to each of a left and a right forearm. For instance, line 178, corresponding to the left forearm indicates a measurement of range of motion of approximately 82 degrees on May 15th and May 17th. Line 178 clearly indicates a dip in range of motion on May 18th to approximately 77 degrees before climbing above 82 degrees on May 20th and 21st and achieving the high point on May 24th with 84 degrees. In some embodiments, a therapist may be able to use this presentation of data to compare and contrast measurements of each arm for each day.

In some embodiments, a chart, such as line chart 1710, may reveal improvement in left forearm pronation, but based on the chart, the therapist is likely to focus on the right forearm pronation. For instance, line 176 indicates limited motion in the right forearm pronation movement and a therapist may choose to work to improve the range of motion based on this indication. Line 176, corresponding to the right forearm indicates measurements of range of motion that start around 38 degrees and increase to 68 degrees. Chart 1710 illustrates an improvement of 30 degrees over a period of nine (9) days. As depicted in chart 1710, even the maximum value of line 1716 is 14 degrees below the range of motion for the left forearm pronation.

FIG. 17A may be used, for instance, to demonstrate progress of a patient's range of motion for an exercise or activity involving forearm pronation. For instance, a VR world task may including virtually twisting a key, turning a doorknob, ladling soup, or other activities featuring rotation of the forearms.

Activities, for example, may involve games and tasks that require patient movement to complete.

FIG. 17B depicts a chart from an exemplary supervisor interface for an illustrative system, in accordance with some embodiments of the disclosure. For instance, FIG. 17B illustrates a user interface featuring a range of motion chart for shoulder extension. Shoulder extension is considered rotating the arm downward or backwards, while shoulder flexion is considered forward lifting of the arm upward around the shoulder, as depicted in FIG. 10D. The interface depicted in FIG. 17B may be from a user interface for use by an operator or therapist after several therapy sessions using a VR system in accordance with one or more embodiments.

FIG. 17B includes line chart 1720 featuring a vertical and a horizontal axis. In line chart 1720, the horizontal axis is date axis 1722 which includes dates ranging from 05/15 to 05/24. In line chart 1720, the vertical axis is angle axis 1724 which includes degrees representing range of motion from about 40 degrees to 90 degrees. In some embodiments, the axes may be scaled differently, as the range of motion for shoulder flexion may be close to 140-180 degrees. In some embodiments, range of motion measurements for each side of the body (e.g., left or right) may be depicted separately.

Line chart 1720 features two lines, line 1726 and line 1728, which correspond to each of a left and a right shoulder. For instance, line 178, corresponding to the left shoulder indicates a measurement of range of motion of approximately 67 degrees from on May 15th to May 24th. Line 178 indicates a small dip in range of motion on May 20th to approximately 66 degrees. In some embodiments, a therapist may be able to use this presentation of data to compare and contrast measurements of each arm for each day.

In some embodiments, a chart, such as line chart 1720, may reveal a small improvement in left shoulder extension, but based on the chart, the therapist is likely to focus on the right shoulder extension. For instance, line 176 indicates limited motion in the right shoulder extension movement and a therapist may choose to work to improve the range of motion based on this indication. Line 176, corresponding to the right shoulder indicates measurements of range of motion that start around 46 degrees and increase to 64 degrees. Chart 1720 illustrates an improvement of 18 degrees over a period of nine (9) days. As depicted in chart 1720, even the maximum value of line 1726 is 3 degrees below the range of motion for the left shoulder extension.

FIG. 17B may be used, for instance, to demonstrate progress of a patient's range of motion for an exercise or activity involving shoulder extension and flexion. For instance, a VR world task may include virtually lifting and setting down objects, pulling a rope, scooping sand/snow, swimming, or other activities featuring flexion and extension of the shoulders. Exemplary Virtual Reality Activities According to the Present Disclosure

Some embodiments may include a variety of experiences that incorporate clinically recognized, existing therapeutic and functional exercises to facilitate motor and cognitive rehabilitation. For instance, games and activities may be used to guide a patient in proper therapeutic experiences. Settings for each experience will involve parameters such as turning on and off avatar features and environmental factors as well as switching between activities.

While using a VR system, a therapist should observe the activities for the patient's safety, as well as evaluate the appropriateness of individual exercises including range of motion (ROM) attempted and any other limb or joint limitations unique to that patient. In some embodiments, observation, supervision, and/or instruction may be performed, e.g., directly or remotely.

A VR therapy game might be “Hide and Seek.” Hide and Seek is an activity that can be used with or without a displayed avatar tracking the patient's upper body because it primarily relies on head movement and visual scanning ability. Hide and Seek may depict a patient's avatar in a nature setting of a VR world with one or more animals that react to the patient's acknowledgement of them. In some embodiments, Hide and Seek may serve as an introductory activity. For instance, such an activity may be an experience the patient first starts with, e.g., giving a chance for the sensors to be placed on the patient's body and then activate visualization of the avatar. Once the sensors have synced, body parts of the patient's avatar may appear. In some embodiments, Hide and Seek may also be a final activity prior to ending the patient's session, providing time for the therapist to remove the sensors and for the patient to visualize overall progress they made during the session, e.g., in the form of virtual “rewards.”

Patients “find” a little penguin by hovering a blue “gaze pointer” on the penguin by turning and rotating their head. The penguin will then disappear and reappear in a different location. The pointer is positioned to represent the patient's upper body vertical midline, itself a useful tool as some patients in neurorehabilitation have lost their sense of body position resulting in “midline shift.” The blue pointer provides a visual, external cue as to their true body midline helping them relearn to position themselves in relation to it. The Hide and Seek exercise encourages visual scanning of their environment, an important functional ability, and cognitive recognition of nameable animals, objects, and environmental locations in their immediate surrounding.

Therapists will have control over the range of locations that animals appear and wait to be found through “difficulty” settings on the tablet. Hide and Seek locations in the world will change and evolve over a number of sessions to provide an experience of logical progression and achievement as the patient continues their course of rehabilitation.

Another therapy game might be “Hot Air Balloon.” Hot Air Balloon may be an introductory activity to help the patient work on core control and strength as well as centering and postural proprioception. By leaning their torso from a sitting position in a certain direction, and holding it there against gravity, they fly a hot air balloon in that same direction. There are a number of objectives the patient can achieve by flying the balloon around, such as knocking apples off a tree and contacting other balloons or clouds. To fly the balloon away and towards them, the patient uses thoracolumbar flexion and extension, and to fly from left to right involves thoracolumbar flexion to the left or right.

A sub-activity called “Balloon Pilot” takes place near the ground. The patient-controlled balloon is tethered to the ground to limit balloon travel and encourage simple torso centering. The patient can pilot the balloon on-tether to nearby interactive objects

A sub-activity called “Bumper Band” takes place halfway up the mountainside. The patient uses trunk extension, flexion, as well as lateral flexion to drive the balloon in an untethered mode to bump other balloons with characters in them, back to the performance stage. Objects, such as bridge components, can also be picked up and carried over to the mountain to help hikers cross gaps.

“Summit Rescue” is a sub-activity that takes place at the peak of the mountain where the player has to steer the balloon to bring hikers which made it to the summit, over to the house stage. Wind driven clouds bump into the balloon and move the balloon arbitrarily which the patient is asked to counteract with trunk movements.

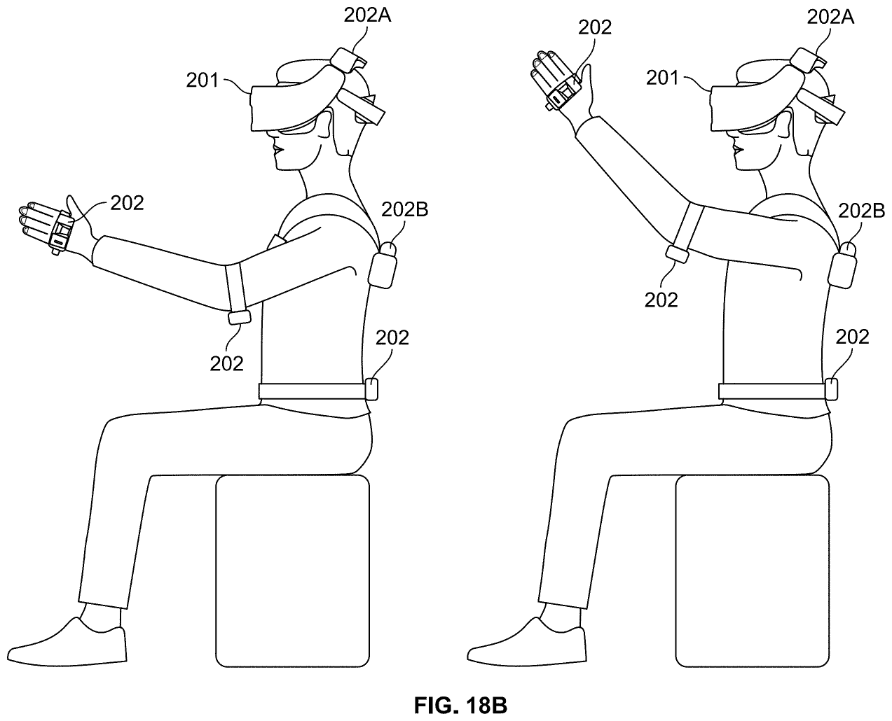

FIG. 18A depicts an exemplary participant interface for an illustrative system, in accordance with some embodiments of the disclosure. For instance, another therapy game may be “Sunrise” as depicted in FIG. 18A. The Sunrise experience is based on simple shoulder flexion. The patient holds their arms out straight in front of them and raises their arms up and over their head in a motion that, ideally, is pure shoulder flexion with a maximum, healthy ROM of 180 degrees. This exercise may be done passively, e.g., with therapist assistance, or actively by the patient themselves.

FIG. 18B is a diagram depicting side views of exemplary activity positions of a participant using an illustrative system, in accordance with some embodiments of the disclosure. For instance, when a shoulder flexion motion is initiated, a Sun character rises from beyond the horizon in proportion to the patient's shoulder flexion ROM. The sun also rotates in the sky and translates side to side, depending on the patient's postural symmetry. When the patient's arms are horizontally and vertically symmetric, and their torso is in vertical alignment with their pelvis and head, the sun will be smiling broadly and high in the sky straight ahead of the patient.

If the patient's posture exhibits asymmetry or other compensating characteristics, the sun's position and the expression on its face will alter from the “ideal” state, thereby providing the patient an external visual cue as to their posture, and allowing them to learn via alternative references, what is proper, non-compensating posture. Maximum shoulder flexion ROM achieved during this experience will be stored as a session output for the therapist's record.

Sunrise may include one or more sub-activities. For instance, as the patient fully lowers and fully raises their arms to the best of their ability, the lighting in the virtual world will exhibit night-time or daytime according to the sun's position, thus greatly accentuating the experience and feedback of a simple coordinated arm raise.

Another sub-activity, the Bumper Crop sub-activity, may involve growing a variety of vegetables by raising and lowering ones arms a number of times in order to trigger the appearance of day-night cycles. Different numbers of cycles necessary to fully grow a vegetable can be controlled by the therapist through difficulty settings. Upon full growth of each vegetable, the patient receives an award that is saved in their game record. This activity may create an incentive for the patient to do multiple repetitions of this exercise if called for by the patient's rehabilitation plan.

An “Ice Cave” sub-activity may involve freeing a variety of Cave Penguins from ice blocks by raising and lowering ones arms a number of times in order to trigger the appearance of day-night cycles. Different numbers of cycles necessary to fully free a penguin can be controlled by the therapist through difficulty settings. Upon each Cave Penguin's release, the patient receives an award that is saved in their game record. This activity creates an incentive for the patient to do multiple repetitions of this exercise if called for by the patient's rehabilitation plan.

Another therapy game may be “Bird Forest.” The Bird Forest experience may incorporate standard functional exercises into a virtual reality experience by requiring the patient to reach out with one or both hands to allow a bird in their immediate vicinity to jump into their hand.

Patients have opportunities to reach from low to high, high to low, from left to right crossing their midline, etc. These exercises mimic standard functional exercises that would be practiced during rehabilitation to help the patient regain skills necessary to live at home with a degree of functional independence, and perform activities such as unpacking groceries, cooking, unloading a dishwasher, self-care, etc.

Adjustable settings include the number of nests and their placement around the patient, whether the hand must properly pronate and supinate in order to pick up and deposit a bird, the smoothness and/or speed of movement required to get a bird to a nest before they fly away, the color of target nests and the patterns applicable to the cognitive exercises.

A sub-activity may be “Free Birds,” where a patient must use their hand(s) to pick up a bird and then move their hand(s) to a nest, also within arm's reach, and maintain that position in order to deposit the bird into the nest. Filling all nests with a bird will reset of the game so it can be played again.

Another sub-activity may be “Nest Hop,” where a patient should use their hand(s) to pick up a bird and move it to a colored target nest in a specific order under time pressure. This sub-activity will exercise both the patient's functional and cognitive ability. When a target nest has been filled, a new target nest will appear, and our patient will have to move the bird from the previous nest to the new target.

In a sub-activity called “Bird Match,” a bird will need to be picked up and dropped off by a patient to colored target nests in a specific order. Only one target nest will be active at a time. The bird will be placed in a sequence of non-repeating colored target nests. When all nests have been filled, the exercise will reset.

Another therapy game may be called “Penguin Sports Park.” In this activity, a patient must move their upper extremities to intersect with an object coming at them, in a time dependent manner. These activities require quick cognitive processing and visual-motor integration to succeed, and thus are more advanced activities for a neurorehabilitation patient.

In a sub-activity called “ChuckleBall™,” a patient fends off approaching Chuckleballs by deflecting them with his head or hands. In some cases, there may be no additional difficulty levels. The Chuckleballs will be kicked continually until a new activity is started.

A sub-activity called “Chuckleball Arena” requires the patient to protect their goal from kicked Chuckleballs coming from the penguin in front of them. Chuckleballs can be deflected by either hand or the head. Depending on the plane of contact of the hand or head, the Chuckleball will deflect in specific directions and advanced patients can learn to deflect the chuckle back into the opposing goal. Other objects and animals in the environment can also serve as targets. The therapist may control how fast the ball travels towards the patient, the distance the patient must reach to block the ball, and the number of balls to be kicked at the patients.

A sub-activity called “Flying Fish” may be similar to Chuckleball where the patient must deflect a fish being pitched at them with the hand or the head. This may elicit a defensive response movement from the patient in VR. Fish may turn from “good” blue fish which are supposed to be deflected to “bad” red spiky fish, which need to be avoided. This requires extra cognitive processing to decide, under time pressure, which fish should be contacted, and which should be avoided, in addition to predicting where the fish are coming and integrating proper movement to accomplish the task.

1. A virtual reality system, the system comprising:
a plurality of sensors comprising a first sensor and a second sensor, the first sensor with a first relative position and the second sensor with a second relative position;
a head-mounted display (HMD) in communication with the plurality of sensors, wherein the HMD is distanced from the first sensor by a first offset; and
processing circuitry configured to:
generate a virtual coordinate system comprising a first virtual position and a head virtual position based on the first offset;
map the first virtual position to the first relative position; and
determine a second virtual position in the virtual coordinate system based on the first virtual position, the first relative position, and the second relative position.

2. The system of claim 1, wherein each of the plurality of sensors has a corresponding relative sensor position and each is configured to wirelessly transmit the corresponding relative sensor position.

3. The system of claim 1, wherein the processing circuitry further configured to determine, for each of the plurality of sensors, a respective virtual position based on each corresponding relative sensor position, the first virtual position, and the first relative position.

4. The system of claim 1, wherein each of the plurality of sensors is configured to transmit the corresponding relative sensor position via radio frequency.

5. The system of claim 1, wherein each corresponding relative sensor position comprises a position and orientation.

6. The system of claim 1, wherein the first sensor is fixed to the HMD.

7. The system of claim 1, wherein a wireless transmitter module (WTM) comprises the second sensor.

8. A method of determining virtual reality world coordinates from relative physical positions of sensors placed on a body, the method comprising:
receiving, by a wireless receiver, a plurality of sensor positions communicated from each of a plurality of sensors placed on the body, the plurality of sensors including a first sensor in spaced relation by a physical offset to a head-mounted display (HMD) and a second sensor;
accessing virtual world coordinates for a virtual world position of the HMD;
determining a virtual world position of the first sensor based on the physical offset and the virtual world position of the HMD;
determining a relative physical position of the second sensor in relation to the first sensor;
determining virtual world position of the second sensor based on the relative physical position of the second sensor and the virtual world position of the first sensor; and
determining a virtual world position for each remaining sensor of the plurality of sensors based on the virtual world position of the second sensor and a relative physical position of each of the plurality of sensors to the second sensor.

9. The method of claim 8, wherein the plurality of sensors further comprises two hand sensors, two elbow sensors, and a hip sensor.

10. The method of claim 8, wherein the receiving is performed wirelessly via radio frequency.

11. The method of claim 8, the method further comprising transmitting to a therapist device at least one virtual world position corresponding to one of the plurality of sensors.

12. A method of assigning sensors placed on a body, the method comprising:
receiving, by a wireless receiver, a plurality of sensor positions communicated from each of a plurality of sensors placed on the body, each of the plurality of sensor positions comprising a height and a hemisphere;
identifying a wireless transmitter module (WTM) sensor of the plurality of sensors placed on the body;
identifying a head sensor of the plurality of sensors based on one of the received plurality of sensor positions being most high;
comparing the position of each of the plurality of sensors to the position of the WTM sensor; and
identifying each of the plurality of sensors based on the position of each of the plurality of sensors in relation to the WTM sensor.

13. The method of claim 12, wherein identifying remaining sensors comprises identifying remaining sensors based on sensor positions indicating a left hemisphere or a right hemisphere.

14. The method of claim 12, wherein identifying remaining sensors comprises identifying an elbow sensor based on a similar height to the WTM sensor.

15. The method of claim 12, wherein identifying remaining sensors includes identifying a hip sensor and two hand sensors based on a similar height to each of the hip sensor and hand sensors.

16. The method of claim 12, wherein identifying remaining sensors includes identifying ankle sensors based on the position of the ankle sensors being lowest.

17. The method of claim 12, wherein the receiving is performed wirelessly via radio frequency.

18. A method of automatically correcting sensor orientation, the method comprising:
receiving a plurality of sensor data communicated from each of a plurality of sensors placed on the body;
generating an avatar skeleton based on the sensor data;
determining an angle for a joint of the avatar skeleton;
comparing the angle of the joint to a predetermined threshold;
in response to determining the angle of the joint is greater than the predetermined threshold, inverting a yaw axis for a corresponding one of the plurality of sensors.

19. The method of claim 18, wherein the joint is a wrist.

20. The method of claim 19, wherein the predetermined threshold is between 50 and 60 degrees.

21. The method of claim 18, wherein the joint is an elbow.

22. The method of claim 21, wherein the predetermined threshold is between 170 and 190 degrees.

23. The method of claim 18, wherein the joint is a hip.

24. The method of claim 23, wherein the predetermined threshold is between 45 and 60 degrees.

25.-51. (canceled)
